SEQUENCE LISTING (18 sequences)

SEQ ID NO: 1 - DNA, 20 bp, synthetic construct
  misc_feature 1..20: note = Chemically synthesized oligonucleotide Myh6-Forward
  source 1..20: mol_type = other DNA
accaacctgtccaagttccg

SEQ ID NO: 2 - DNA, 20 bp, synthetic construct
  misc_feature 1..20: note = Chemically synthesized oligonucleotide Myh6-Reverse
  source 1..20: mol_type = other DNA
tattggccacagcgagggtc

SEQ ID NO: 3 - DNA, 21 bp, synthetic construct
  misc_feature 1..21: note = Chemically synthesized oligonucleotide CD31-Forward
  source 1..21: mol_type = other DNA
agctgctccacttctgaactc

SEQ ID NO: 4 - DNA, 20 bp, synthetic construct
  misc_feature 1..20: note = Chemically synthesized oligonucleotide CD31-Reverse
  source 1..20: mol_type = other DNA
tcaagggaggacacttccac

SEQ ID NO: 5 - DNA, 20 bp, synthetic construct
  misc_feature 1..20: note = Chemically synthesized oligonucleotide Col3a1-Forward
  source 1..20: mol_type = other DNA
ggtgacagaggagaaactgg

SEQ ID NO: 6 - DNA, 20 bp, synthetic construct
  misc_feature 1..20: note = Chemically synthesized oligonucleotide Col3a1-Reverse
  source 1..20: mol_type = other DNA
atgtggtccaactggtcctc

SEQ ID NO: 7 - DNA, 20 bp, synthetic construct
  misc_feature 1..20: note = Chemically synthesized oligonucleotide B2M-Forward
  source 1..20: mol_type = other DNA
ctcggtgaccctggtctttc

SEQ ID NO: 8 - DNA, 20 bp, synthetic construct
  misc_feature 1..20: note = Chemically synthesized oligonucleotide B2M-Reverse
  source 1..20: mol_type = other DNA
ttcagtatgttcggcttccc

SEQ ID NO: 9 - DNA, 23 bp, synthetic construct
  misc_feature 1..23: note = Chemically synthesized oligonucleotide eGFP-X
  source 1..23: mol_type = other DNA
ggttccctagttagccagagagc

SEQ ID NO: 10 - DNA, 22 bp, synthetic construct
  misc_feature 1..22: note = Chemically synthesized oligonucleotide eGFP-Y
  source 1..22: mol_type = other DNA
gagtgcttcaagtagtgtgtgc

SEQ ID NO: 11 - DNA, 26 bp, synthetic construct
  misc_feature 1..26: note = Chemically synthesized oligonucleotide eGFP-M
  source 1..26: mol_type = other DNA
agcagatcttgtcttcgttgggagtg

SEQ ID NO: 12 - DNA, 24 bp, synthetic construct
  misc_feature 1..24: note = Chemically synthesized oligonucleotide eGFP-Z
  source 1..24: mol_type = other DNA
ccgtctgttgtgtgactctggtaa

SEQ ID NO: 13 - DNA, 26 bp, synthetic construct
  misc_feature 1..26: note = Chemically synthesized oligonucleotide eGFP-F
  source 1..26: mol_type = other DNA
cattggtcttaaaggtaccgagctcg

SEQ ID NO: 14 - DNA, 26 bp, synthetic construct
  misc_feature 1..26: note = Chemically synthesized oligonucleotide eGFP-L
  source 1..26: mol_type = other DNA
gatccctcagacccttttagtcagtg

SEQ ID NO: 15 - protein, 5 aa, unidentified
  REGION 1..5: note = enterokinase cleavage site
DDDDK

SEQ ID NO: 16 - DNA, 609 bp, synthetic construct
  misc_feature 1..609: note = Mus sp. myocyte viral integrant site
  misc_difference 2: note = n is a, c, g, or t
  source 1..609: mol_type = other DNA
gntagaattcgccccttcattggtcttaaggtaccgagctcgaagatcatgggacagtgcccatgtctgcttctgttgacagtggccatgacctgtttcctggaggacaacagcctgtagctaagttaaggtcctttaagcttgtatccacctacatttaactgggatgagacattagatactgcaaattttgaatttaatttccatcccaggaactccacactttttattctgtcacaacgtttgcgacctgttacttgggtaataaagaatatcttctttaaaaaaagaaaaagacctggtagaacatccgaaggcctgtctgcagaaggtagattttaaaaaaagcctggcatacaagaatagatgtgtaactaccacgcctgtggcagcacctgctaggcaggagaccacaggaagccagcctctttgttctgctcaggccacttcctagtccttgctattcagaataatgatgttacaggtgggccccgctgcccttctgcagctccctaccaccccagggtgatcctcagatgcaccctgagctgttggtggctgctagagattttccacactgactaaaagggtctgagggatcaagggcga

SEQ ID NO: 17 - DNA, 300 bp, synthetic construct
  misc_feature 1..300: note = Mus sp. endothelial viral integrant site
  misc_difference 2: note = n is a, c, g, or t
  source 1..300: mol_type = other DNA
gngttacgaattcgcccttcattgggtcttaaggtaccgagctcgaagttggcatattggtacacacctctaaagccagaatttggaagttagaaacaggcgaatccgcatgagactgaagccagcctgatctagacagtgacttctagtccagctagagctagtaagaagaccctatctttcttaaatacatatacaaacaaacaaacatacaatagtacactgctagagattttccacactgactaaaaggtctgagggatcaagggcgaattcgcggccgctaaattcaattcgccc

SEQ ID NO: 18 - DNA, 415 bp, synthetic construct
  misc_feature 1..415: note = Mus sp. myocyte viral integrant site
  source 1..415: mol_type = other DNA
gcgcgattcgcccttcattggtcttaaaggtaccgagctcgatggttgtgagccaccaccatgtggttgctgggatttgaactccagacctttggaagagcagtcgggtgctcttactcactgagccatctcaccagctcaaagattttattataatggtttgattgttaccccgtcttgtgctgagatatgtgataactgatcaactccaggacttttatacaaaaggaatctggacttgccctcagcagtcatagatggattaaatgttacacatcacacgtatatttggctgacttaaaacttggctttgggaattgctagagattttccacactgactaaaagggtctgagggatcaagggcgaattcgtttaaacctgcaggactagtccctttagtgagggttaattct

(19) United States
(12) Patent Application Publication
(10) Pub. No.: US 2023/0256022 A1
(43) Pub. Date: Aug. 17, 2023

(54) C-KIT-POSITIVE BONE MARROW CELLS AND USES THEREOF

(71) Applicant: AAL SCIENTIFICS, INC., New York, NY (US)

(72) Inventors: Piero ANVERSA, New York, NY (US); Annarosa LERI, New York, NY (US)

(73) Assignee: AAL SCIENTIFICS, INC., New York, NY (US)

(21) Appl. No.: 18/084,206

(22) Filed: Dec. 19, 2022

Related U.S. Application Data
(62) Division of application No. 16/481,587, filed on Jul. 29, 2019, now abandoned.
(60) Provisional application No. 62/453,428, filed on Feb. 1, 2017.

(51) Int. Cl. A61K 35/28; C12N 5/077; C12N 5/0775

(52) U.S. Cl. CPC A61K 35/28; C12N 5/0657; C12N 5/0663; C12N 2501/727

(57) ABSTRACT

Disclosed herein are compositions comprising myogenic bone marrow cells that are c-kit positive. Such compositions are useful for treating cardiac diseases or disorders. Also disclosed herein are methods of producing myogenic bone marrow cells are c-kit positive. Further disclosed are cardiopoietic genes having enhanced expression in c-kit positive myogenic bone marrow cells.

### CROSS-REFERENCE TO RELATED APPLICATIONS

This application is a division of U.S. application Ser. No. 16/481,587, filed Jul. 29, 2019. Application Ser. No. 16/481,587 is a United States national stage entry of an International Application serial no. PCT/US2018/016483 filed Feb. 1, 2018 which claims priority to U.S. Provisional Patent Application Ser. No. 62/453,428 filed Feb. 1, 2017. The contents of these applications are incorporated herein by reference in their entirety.

### STATEMENT OF GOVERNMENT INTEREST

This invention was made with government support under Grant No. NHLBI/R01HL65577 awarded by the National Institutes of Health. The government has certain rights in the invention.

### REFERENCE TO SEQUENCE LISTING XML SUBMITTED ELECTRONICALLY

This application contains a Sequence Listing which has been submitted in XML format via PatentCenter and is hereby incorporated by reference in its entirety. The Sequence Listing XML file, created Dec. 19, 2022, is named “55453-007-DIV_SEQUENCE_LISTING” and has a size of 26,624 bytes.

### FIELD OF THE INVENTION

The present invention relates generally to the field of cardiology. More specifically, the invention relates to myogenic bone marrow cells are c-kit positive and the use of such bone marrow cells to treat or prevent heart diseases or disorders.

### BACKGROUND OF THE INVENTION

A major biological controversy of the last decade has involved the plasticity of c-kit-positive bone marrow cells (c-kit-BMCs) and their ability to form cell-lineages different from the organ of origin.¹ The possibility that c-kit-BMCs can form cardiomyocytes and coronary vessels repairing the injured heart experimentally² was accepted with enthusiasm by cardiologists, resulting in the clinical implementation of bone marrow mononuclear cells (BM-MNCs) in patients with myocardial infarction.³ However, a series of negative animal studies challenging the original observations⁴ has shifted the view in the scientific community; even the supporters of the therapeutic efficacy of BM-MNCs have questioned the concept of cell transdifferentiation.

There is a need for improved compositions and methods related to bone marrow cells for the treatment of heart disease.

### SUMMARY OF THE INVENTION

In one embodiment, the invention provides a method of treating or preventing a heart disease or disorder in a subject in need thereof comprising administering isolated myogenic bone marrow cells to the subject, wherein the myogenic bone marrow cells are c-kit positive (c-kit-BMCs). In some embodiments, the heart disease or disorder is heart failure, diabetic heart disease, rheumatic heart disease, hypertensive heart disease, ischemic heart disease, cerebrovascular heart disease, inflammatory heart disease and/or congenital heart disease. In some embodiments, the c-kit-BMCs are a subpopulation of c-kit positive bone marrow cells isolated from bone marrow. In some embodiments, the c-kit-BMCs are able to transdifferentiate into cardiomyocytes, endothelial cells, fibroblasts, coronary vessels and/or cells of mesodermal origin. In some embodiments, the c-kit-BMCs have enhanced expression of cardiopoietic genes compared to non-myogenic c-kit positive bone marrow cells. In some embodiments, the c-kit-BMCs have enhanced expression of RYR3, OSM, Jag1, Hey2 and Smyd3 compared to non-myogenic c-kit positive bone marrow cells.

In one embodiment, the invention provides a method of repairing and/or regenerating damaged tissue of a heart in a subject in need thereof comprising: (a) extracting c-kit positive bone marrow cells from bone marrow; (b) selecting myogenic c-kit positive bone marrow cells (c-kit-BMCs) from step (a); (c) culturing and expanding said c-kit-BMCs from step (b); and (d) administering a dose of said c-kit-BMCs from step (c) to an area of damaged tissue in the subject effective to repair and/or regenerate the damaged tissue of the heart. The selecting step may comprise selecting c-kit-BMCs having enhanced expression of RYR3, OSM, Jag1, Hey2 and Smyd3.

In one embodiment, the invention provides a method of producing myogenic c-kit positive bone marrow cells (c-kit-BMCs), comprising: (a) isolating c-kit positive bone marrow cells from bone marrow; (b) selecting myogenic c-kit positive bone marrow cells (c-kit-BMCs) from step (a); and (c) culturing and expanding the c-kit-BMCs of step (b), thereby producing c-kit-BMCs. The selecting step may comprise selecting c-kit-BMCs having enhanced expression of RYR3, OSM, Jag1, Hey2 and Smyd3.

In one embodiment, the invention provides a pharmaceutical composition comprising a therapeutically effective amount of myogenic c-kit positive bone marrow cells (c-kit-BMCs) and a pharmaceutically acceptable carrier for repairing and/or regenerating damaged tissue of a heart.

In one embodiment, the invention provides a composition comprising myogenic c-kit positive bone marrow cells (c-kit-BMCs). In one embodiment, the c-kit-BMCs express RYR3, OSM, Jag1, Hey2 and Smyd3.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, and 1D: c-kit-BMCs acquire distinct cardiac cell phenotypes in vivo. FIG. 1A, Scatter plots illustrating the strategy for cardiac cell isolation based on the expression of c-kit, Thy1.2 and CD31. CTRL: isotype control; SSC: side scatter. FIG. 1B, Isolated cardiomyocytes expressing α-sarcomeric actin (α-SA, red), ECs expressing von Willebrand factor (vWF, yellow) and fibroblasts expressing procollagen (Pro-Col, green). FIG. 1C, Transcripts for α-myosin heavy chain (Myh6), c-kit, CD31, collagen type III a-1 (Col3a1), and [3-2 microglobulin (B2M) in isolated cardiomyocytes (Myo), c-kit-BMCs (c-kit), ECs and fibroblasts (Fbl). Myocardium (MC) was used as control. bp: base pairs. FIG. 1D, The PCR products correspond to the sites of integration of the viral genome in the DNA of c-kit-BMCs and myocytes. The upper band shows the pCR4-TOPO TA vector.

FIGS. 2A, 2B, 2C, 2D, 2D, and 2E: c-kit-BMCs express three fluorescent reporter genes in vitro. FIG. 2A and FIG. 2B, Low power magnification images (A) illustrating native fluorescence of cultured c-kit-BMCs transduced with three lentiviruses carrying eCFP (blue), mCherry (red) or eYFP (yellow). Arrows indicate the cells illustrated at higher magnification in panel B where individual c-kit-BMCs show the primary colors, i.e., red, yellow and cyan, and their multiple combinations. FIG. 2C and FIG. 2D, Scatter plots documenting the detection of YFP, CFP, or mCherry and their combinations in c-kit-positive cells by flow-cytometry. Non-infected c-kit-BMCs were used as negative control. FIG. 2E, The color chart illustrates the proportion of c-kit-BMCs labeled by multiple colors. The fraction of unlabeled cells is also indicated.

FIGS. 3A, 3B, 3C: c-kit-BMCs regenerate the infarcted myocardium. These images were collected 4 to 7 days after infarction and cell delivery. FIG. 3A, Below a thin layer of spared endomyocardium (EM), the infarcted region is replaced by a large number of small fluorescently labeled cells. A cocktail of anti-mCherry and anti-CFP was employed to identify the progeny of c-kit-BMCs (green). In the EM, cardiomyocytes are positive for troponin I (TnI; red). FIG. 3B and FIG. 3C, A cocktail of anti-mCherry, anti-YFP and anti-CFP was employed to identify the progeny of c-kit-BMCs (green). Small newly-formed cells (green), at times positive for GAT A4 (B) and Nkx2.5 (C) are present between spared card iomyocytes positive for α-sarcomeric actin (α-SA, gray-white). Two of these cells included in the squares are shown at higher magnification in the insets. In panel B, the inset illustrates, on the left, a cell positive for the fluorescent tag (green) and GATA4 (red dots in the nucleus) and, on the right, the same cell expressing α-SA (gray-white). In panel C, the inset illustrates, on the left, a cell positive for the fluorescent tag (green) and Nkx2.5 (red dots in the nucleus) and, on the right, the same cell expressing α-SA (gray-white).

FIGS. 4A-4B. c-kit-BMCs acquire the cardiomyocyte lineage. FIG. 4A, The regenerated cells in the lower part of the left panel are included in a rectangle; these cells are tagged by YFP (green) and CFP (blue); green and blue together=turquoise. Labeling for α-SA (red), YFP and CFP is shown separately in the right three panels. FIG. 4B, Group of developing cardiomyocytes labeled in two consecutive sections to detect, separately, the three tags: YFP (green) and CFP (blue) and their combination (turquoise). The upper left panel shows the co-localization of α-SA (red), YFP (green) and CFP (blue), and the upper right panel shows the co-localization of α-SA (red) and mCherry (assigned color: green). The lower two panels illustrate the same images with nuclei stained by DAPI (white).

FIGS. 5A, 5B, AND 5C: c-kit-BMCs expand clonally and regenerate the infarcted myocardium. A cocktail of anti-mCherry, anti-YFP, and anti-CFP was employed to identify the progeny of c-kit-BMCs (green). FIG. 5A, At 21 days, the infarcted myocardium is almost completely replaced by newly-formed small cells (green). As examples, the cells pointed by the two yellow arrowheads are illustrated at higher magnification in the insets (right four small panels) where the co-localization of GATA4 (red) and α-SA (white) is apparent. EM: endomyocardium. FIGS. 5B and 5C, Two other examples in which mCherry, YFP and CFP positive cells (green; left panels) express GATA4 (red) and α-SA (white; right panels).

FIGS. 6A, 6B, AND 6C: The integration of regenerated cardiomyocytes is coupled with improved LV function. FIGS. 6A and 6B, A cocktail of anti-mCherry, anti-YFP, and anti-CFP was employed to identify the progeny of c-kit-BMCs (green). FIG. 6A, Connexin 43 (Cx43, red) is expressed at the interface of newly-formed myocytes (mCherry-YFP-CFP, green; α-SA, white) and recipient myocytes, as pointed by yellow arrows and arrowheads. As examples, the structures indicated by the three yellow arrows are shown at higher magnification in the insets. The insets illustrate first mCherry, YFP and CFP (green), together with Cx43, and then the localization of α-SA (white) and Cx43 (arrows). FIG. 6B, N-cadherin (N-Cadh, red) is detected between regenerated and spared cardiomyocytes (yellow arrows and arrowheads). As examples, the structures indicated by the three yellow arrows are shown in the insets (arrows). FIG. 6C, Measurements of ventricular pressures and dP/dt in untreated infarcts (MI: n=11) and cell-treated infarcts (Ml+BMCs: n=8). *P<0.05.

FIGS. 7A, 7B, AND 7C: Myogenic and non-myogenic clonal c-kit-BMCs. FIG. 7A, Sorted GFP-positive-c-kit-BMCs, plated at limiting dilution in semi-solid medium, generate single cell-derived clones (upper panels, phase contrast micrographs; lower panels, native GFP fluorescence). FIG. 7B, Scatter plots of c-kit and GFP expression in clonal c-kit-BMCs. The number in the boxes corresponds to the sampled clones. FIG. 7C, Three weeks after myocardial infarction and injection of clonal GFP-positive-c-kit-BMCs, sites of viral integrations were detected in aliquots of the delivered cells and in isolated regenerated cardiomyocytes. The PCR products correspond to the sites of integration of the viral genome in the DNA of c-kit-BMCs and cardiomyocytes.

FIG. 8. Detection of integration sites. Common insertion sites were identified by PCR and sequencing in c-kit-BMCs and cardiomyocytes, and were color-coded.

FIG. 9 depicts a schematic of the PCR-based protocol employed for the detection of the sites of lentiviral integration in the genome of c-kit-BMCs.

FIGS. 10A, 10B, AND 10C. Sequence analysis of PCR products. Examples of DNA sequences comprising the viral (green line) and mouse (black line) genome. The magenta line corresponds to Taq I digestion site. SEQ ID NO: 16 is shown in FIG. 10A, SEQ ID NO: 17 is shown in FIG. 10B, and SEQ ID NO: 18 is shown in FIG. 10C.

FIGS. 11A-11B. Lentiviral integration in the DNA of c-kit-BMCs acquiring distinct cardiac cell phenotypes in vivo. FIGS. 11A, Chromosome number, length of key DNA sequences and the closest gene to the integration site are listed. FIGS. 11B, Sites of integration (IS) of the viral genome in the myocardium of different mice: myocytes (red dots), ECs (blue dots), fibroblasts (yellow dots) and c-kit-BMCs (green dots). In animal number 6 no sites of integration were found.

FIGS. 12A, 12B, 12C, and 12D: Engrafted c-kit-BMCs and their progeny express the three fluorescent reporter genes in vivo after infarction. Four days after infarction and the delivery of c-kit-BMCs transduced with the 3 lentiviruses, an area of the infarcted myocardium is replaced by cells positive for mCherry (FIG. 12A, red), YFP (FIG. 12B, green), and CFP (FIG. 12C, blue). These areas were detected by epifluorescence microscopy. The 4 rectangles in the merge panel (FIG. 12D) delineate clusters of cells uniformly labeled: clusters 1 and 2 are composed of cells predominantly white (red, green and blue together=white); cluster 3 is composed of cells predominantly yellow (red and green together=yellow); and cluster 4 is composed of cells predominantly turquoise (green and blue together=turquoise).

FIGS. 13A, 13B, 13C, 13D, 13E, 13F, and 13G: Differentiation of c-kit-BMCs into cardiomyocytes. FIGS. 13A, 13B, 13C, 13D, 13E, and 13F, At 21 days after infarction, newly-formed myocytes and spared myocytes are positive for α-SA (FIG. 13A: red). Nuclei are stained by DAPI (white). BZ: Border zone. The regenerated myocytes are labeled by YFP (green) and CFP (blue) (FIG. 13B), or by YFP, CFP and α-SA (red) (FIG. 13C), or by YFP, CFP, α-SA and DAPI (white) (FIG. 13D). Consecutive sections are shown in FIG. 13E and FIG. 13F. The regenerated myocytes are positive for α-SA (red) (FIG. 13E), for mCherry (red), YFP (green) and CFP (blue) (FIG. 13F). Labeling of DAPI (white) for panel F is shown in the right image (FIG. 13G).

FIGS. 14A-14B. Differentiation of c-kit-BMCs into coronary vessels. FIG. 14A, Small vessels defined by an endothelial lining labeled by YFP (green) and CD31 (red; arrows). Two of these vessels (yellow arrows) are illustrated at higher magnification in the insets (right panels) where the individual channels for YFP and CD31 are shown. White arrowheads point to cells positive for both YFP and CD31. FIG. 14B, Coronary arterioles (yellow arrows) were stained by a cocktail of mCherry, YFP and CFP (green). Endothelial cells are positive for CD31 (red) and smooth muscle cells (SMCs) for α-SMA (blue). Two of these arterioles (yellow arrows) are illustrated at higher magnification in the insets (right panels) where the individual channels for mCherry-YFP-CFP (green), CD31 (red) and α-SMA (blue) are shown.

### DETAILED DESCRIPTION OF THE INVENTION

C-kit positive bone marrow cells constitute a critically important hematopoietic stem cell class. Certain embodiments described herein are based on the discovery that a subpopulation of these cells has the intrinsic ability to cross lineage boundaries and commit to the cardiac fate. In some embodiments, myogenic, c-kit positive bone marrow cells (c-kit-BMCs) are useful for therapeutic purposes. In some embodiments, c-kit-BMCs are able to transdifferentiate into cardiomyocytes, endothelial cells, fibroblasts, coronary vessels and/or cells of mesodermal origin. In some embodiments, c-kit-BMCs have enhanced expression of cardiopoietic genes compared to non-myogenic c-kit positive bone marrow cells. In certain embodiments, cardiopoietic genes include RYR3, OSM, Jag1, Hey2 and Smyd3.

Two single-cell-based approaches, viral gene-tagging and multicolor clonal-marking, were employed to define the functional heterogeneity of c-kit-BMCs. Described herein are mouse c-kit-BMCs that engraft within the infarcted myocardium, expand clonally and differentiate into myocardial structures, restoring partly the integrity of the organ. Newly-formed cardiomyocytes, endothelial cells, fibroblasts and c-kit-BMCs showed common sites of viral integration in their genome providing strong evidence in favor of BMC transdifferentiation. Additionally, myogenic c-kit-BMCs self-renewed in vivo and may have a long-term effect on the recovery of the infarcted heart. To determine the molecular signature of c-kit-BMCs capable of generating cardiomyocytes, clonal cells, derived from growth of individual c-kit-BMCs, were delivered to the injured heart and based on their ability to form cardiomyocytes their transcriptional profile was defined by RNA sequencing. Five highly-scored myocyte-related genes were identified in myogenic c-kit-BMCs: ryanodine receptor 3, Oncostatin M, Jagged1, Hey2, and SET-dependent-methyltransferase-3. Importantly, myogenic and non-myogenic c-kit-BMCs expressed a variety of cytokines, documenting their potential paracrine effect on the myocardium. A class of c-kit-BMCs disclosed herein is characterized by a network of cardiopoietic genes that support the proficiency of these cells to home to the infarcted myocardium and acquire the cardiomyocyte fate.

In some embodiments, the invention provides a population of isolated adult myogenic c-kit-BMCs. In some embodiments, a population of adult c-kit-BMCs comprises at least 90%, at least 93%, at least 95%, at least 97%, at least 98% or at least 99% myogenic adult c-kit-BMCs BMCs have enhanced expression of cardiopoietic genes (e.g., include RYR3, OSM, Jag1, Hey2 and Smyd3) compared to non-myogenic c-kit positive bone marrow cells.

In one embodiment, the invention provides a pharmaceutical composition comprising a therapeutically effective amount of myogenic c-kit positive bone marrow cells (c-kit-BMCs) and a pharmaceutically acceptable carrier for repairing and/or regenerating damaged tissue of a heart.

In one embodiment, the invention provides a composition comprising myogenic c-kit positive bone marrow cells (c-kit-BMCs). In one embodiment, the c-kit-BMCs express RYR3, OSM, Jag1, Hey2 and Smyd3.

In one embodiment, the invention provides a method of treating or preventing a heart disease or disorder in a subject in need thereof comprising administering isolated myogenic bone marrow cells to the subject, wherein the myogenic bone marrow cells are c-kit positive (c-kit-BMCs). In some embodiments, the heart disease or disorder is heart failure, diabetic heart disease, rheumatic heart disease, hypertensive heart disease, ischemic heart disease, cerebrovascular heart disease, inflammatory heart disease and/or congenital heart disease. In some embodiments, the c-kit-BMCs are a subpopulation of c-kit positive bone marrow cells isolated from bone marrow. In some embodiments, the c-kit-BMCs are able to transdifferentiate into cardiomyocytes, endothelial cells, fibroblasts, coronary vessels and/or cells of mesodermal origin. In some embodiments, the c-kit-BMCs have enhanced expression of cardiopoietic genes compared to non-myogenic c-kit positive bone marrow cells. In some embodiments, the c-kit-BMCs have enhanced expression of RYR3, OSM, Jag1, Hey2 and Smyd3 compared to non-myogenic c-kit positive bone marrow cells.

In one embodiment, the invention provides a method of repairing and/or regenerating damaged tissue of a heart in a subject in need thereof comprising: (a) extracting c-kit positive bone marrow cells from bone marrow; (b) selecting myogenic c-kit positive bone marrow cells (c-kit-BMCs) from step (a); (c) culturing and expanding said c-kit-BMCs from step (b); and (d) administering a dose of said c-kit-BMCs from step (c) to an area of damaged tissue in the subject effective to repair and/or regenerate the damaged tissue of the heart. The selecting step may comprise selecting c-kit-BMCs having enhanced expression of RYR3, OSM, Jag1, Hey2 and Smyd3.

In some embodiments, c-kit-BMCs can repair damaged heart tissue in diabetic mice. Examples of mouse models of diabetes and methods of implanting stem cells in such mice are described in e.g., Hua et al., PLoS One, 2014 Jul. 10; 9(7):e102198. When c-kit-BMCs are placed into a mouse with a damaged heart, long-term engraftment of the administered c-kit-BMCs can occur, and these c-kit-BMCs can differentiate into, for example, endothelial cells, fibroblasts, coronary vessels and/or cells of mesodermal origin, which can lead to subsequent heart tissue regeneration and repair. The mouse experiments can indicate whether isolated c-kit-BMCs can be used for heart tissue regeneration for treatment of, e.g, ischemic cardiomyopathy, heart failure or diabetic heart disease in human patients. Accordingly, provided herein are methods for the treatment and/or prevention of a heart disease or disorder in a subject in need thereof.

In some embodiments, a subject treated by the methods and compositions described herein has a heart disease or disorder. As used herein, the term “heart disease or disorder”, “heart disease”, “heart condition” and “heart disorder” are used interchangeably. Heart diseases and/or conditions can include heart failure, diabetic heart disease, rheumatic heart disease, hypertensive heart disease, ischemic heart disease, cerebrovascular heart disease, inflammatory heart disease and/or congenital heart disease. In some embodiments, a subject treated by the methods or compositions described herein has type 1 diabetes or type 2 diabetes. The methods described herein can be used to treat, ameliorate the symptoms, prevent and/or slow the progression of a number of heart diseases or disorders or their symptoms. In some embodiments of all aspects of the therapeutic methods described herein, a subject having a heart disease or disorder is first selected prior to administration of the recombinant myogenic c-kit-BMCs.

The terms “subject”, “patient” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or food production mammal, e.g., cow, sheep, pig, and the like.

Accordingly, in some embodiments of the therapeutic methods described herein, a subject is a recipient subject, i.e., a subject to whom the myogenic c-kit-BMCs described herein are being administered, or a donor subject, i.e., a subject from whom a heart tissue sample comprising myogenic c-kit-BMCs described herein is being obtained. A recipient or donor subject can be of any age. In some embodiments, the subject is a “young subject,” defined herein as a subject less than 10 years of age. In other embodiments, the subject is an “infant subject,” defined herein as a subject is less than 2 years of age. In some embodiments, the subject is a “newborn subject,” defined herein as a subject less than 28 days of age. In one embodiment, the subject is a human adult. In one embodiment of all aspects of the compositions and methods described, the myogenic c-kit-BMCs are allogeneic.

The isolated myogenic c-kit-BMCs described herein can be administered to a selected subject having any heart disease or disorder or predisposed to developing a heart disease or disorder. The administration can be by any appropriate route which results in an effective treatment in the subject. In some aspects of these methods, a therapeutically effective amount of isolated myogenic c-kit-BMCs described herein is administered through vessels, directly to the tissue, or a combination thereof. Some of these methods involve administering to a subject a therapeutically effective amount of isolated myogenic c-kit-BMCs described herein by injection, by a catheter system, or a combination thereof.

As used herein, the terms “administering,” “introducing”, “transplanting” and “implanting” are used interchangeably in the context of the placement of cells, e.gmyogenic c-kit-BMCs of the invention into a subject, by a method or route which results in at least partial localization of the introduced cells at a desired site, such as a site of injury or repair, such that a desired effect(s) is produced. The cells, e.g., myogenic c-kit-BMCs, or their differentiated progeny (e.g., cardiomyocytes, endothelial cells, fibroblasts, coronary vessels and/or cells of mesodermal origin) can be implanted directly to the heart, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of all aspects of the therapeutic methods described herein, an effective amount of a population of isolated myogenic c-kit-BMCs is administered directly to the heart of an individual suffering from heart disease by direct injection. In other embodiments of all aspects of the therapeutic methods described herein, the population of isolated myogenic c-kit-BMCs is administered via an indirect systemic route of administration, such as a catheter-mediated route.

One embodiment of the invention includes use of a catheter-based approach to deliver the injection. The use of a catheter precludes more invasive methods of delivery such as surgically opening the body to access the heart. As one skilled in the art is aware, optimum time of recovery would be allowed by the more minimally invasive procedure, which as outlined here, includes a catheter approach. When provided prophylactically, the isolated myogenic c-kit-BMCs can be administered to a subject in advance of any symptom of a heart disease or disorder. Accordingly, the prophylactic administration of an isolated myogenic c-kit-BMCs population serves to prevent a heart disease or disorder, or further progress of heart diseases or disorders as disclosed herein.

When provided therapeutically, isolated myogenic c-kit-BMCs are provided at (or after) the onset of a symptom or indication of a heart disease or disorder, or for example, upon the onset of diabetes.

As used herein, the terms “treat,” “treatment,” “treating,” or “amelioration” refer to therapeutic treatment, wherein the object is to reverse, alleviate, ameliorate, decrease, inhibit, or slow down the progression or severity of a condition associated with a disease or disorder. The term “treating” includes reducing or alleviating at least one adverse effect or symptom of a condition, disease or disorder associated with a heart disease). Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. In some embodiments, “treatment” and “treating” can also mean prolonging survival of a subject as compared to expected survival if the subject did not receive treatment.

As used herein, the term “prevention” refers to prophylactic or preventative measures wherein the object is to prevent or delay the onset of a disease or disorder, or delay the onset of symptoms associated with a disease or disorder. In some embodiments, “prevention” refers to slowing down the progression or severity of a condition or the deterioration of cardiac function associated with a heart disease or disorder.

In another embodiment, “treatment” of a heart disease or disorder also includes providing relief from the symptoms or side-effects of the disease (including palliative treatment). In some embodiments of the aspects described herein, the symptoms or a measured parameter of a disease or disorder are alleviated by at least 5%, at least 10%, at least 20%, at least 30%, at least 40%, at least 50%, at least 60%, at least 70%, at least 80%, or at least 900/o, upon administration of a population of isolated myogenic c-kit-BMCs, as compared to a control or non-treated subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions as disclosed herein will be decided by the attending physician within the scope of sound medical judgment. The exact amount required will vary depending on factors such as the type of heart disease or disorder being treated, degree of damage, whether the goal is treatment or prevention or both, age of the subject, the amount of cells available, etc. Thus, one of skill in the art realizes that a treatment may improve the disease condition, but may not be a complete cure for the disease.

In one embodiment of all aspects of the therapeutic methods described, the term “effective amount” as used herein refers to the amount of a population of myogenic c-kit-BMCs needed to alleviate at least one or more symptoms of the heart disease or disorder, and relates to a sufficient amount of pharmacological composition to provide the desired effect, e.g., treat a subject having heart disease. The term “therapeutically effective amount” therefore refers to an amount of isolated myogenic c-kit-BMCs using the therapeutic methods as disclosed herein that is sufficient to effect a particular effect when administered to a typical subject, such as one who has or is at risk for heart disease.

In another embodiment of all aspects of the methods described, an effective amount as used herein would also include an amount sufficient to prevent or delay the development of a symptom of the disease, alter the course of a disease symptom (for example, but not limited to, slow the progression of a symptom of the disease), or even reverse a symptom of the disease. The effective amount of myogenic c-kit-BMCs needed for a particular effect will vary with each individual and will also vary with the type of heart disease or disorder being addressed. Thus, it is not possible to specify the exact “effective amount”. However, for any given case, an appropriate “effective amount” can be determined by one of ordinary skill in the art using routine experimentation.

In some embodiments of all aspects of the therapeutic methods described, the subject is first diagnosed as having a disease or disorder affecting the heart prior to administering the myogenic c-kit-BMCs according to the methods described herein. In some embodiments of all aspects of the therapeutic methods described, the subject is first diagnosed as being at risk of developing a heart disease or disorder prior to administering the myogenic c-kit-BMCs, e.g., an individual with a genetic disposition for heart disease or diabetes or who has close relatives with heart disease or diabetes.

For use in all aspects of the therapeutic methods described herein, an effective amount of isolated myogenic c-kit-BMCs comprises at least 10², at least 5×10², at least 10³, at least 5×10³, at least 10⁴, at least 5×10⁴, at least 10⁵, at least 2×10⁵, at least 3×10⁵, at least 4×10⁵, at least 5×10⁵, at least 6×10⁵, at least 7×10⁵, at least 8×10⁵, at least 9×10⁵, or at least 1×10⁶ myogenic c-kit-BMCs or multiples thereof per administration. In some embodiments, more than one administration of isolated myogenic c-kit-BMCs is performed to a subject. The multiple administration of isolated myogenic c-kit-BMCs can take place over a period of time. The myogenic c-kit-BMCs can be generated from BMCs isolated from one or more donors, or from BMCs obtained from an autologous source.

Exemplary modes of administration of myogenic c-kit-BMCs and other agents for use in the methods described herein include, but are not limited to, injection, infusion, inhalation (including intranasal), ingestion, and rectal administration. “Injection” includes, without limitation, intravenous, intraarterial, intraductal, direct injection into the tissue intraventricular, intracardiac, transtracheal injection and infusion. The phrases “parenteral administration” and “administered parenterally” as used herein, refer to modes of administration other than enteral and topical administration, usually by injection, and includes, without limitation, intravenous, intraventricular, intracardiac, transtracheal injection and infusion. In some embodiments, myogenic c-kit-BMCs can be administered by intravenous, intraarterial, intraductal, or direct injection into tissue, or through injection in the liver.

In some embodiments of all aspects of the therapeutic methods described, an effective amount of isolated myogenic c-kit-BMCs is administered to a subject by injection. In other embodiments, an effective amount of isolated myogenic c-kit-BMCs is administered to a subject by a catheter-mediated system. In other embodiments, an effective amount of isolated myogenic c-kit-BMCs is administered to a subject through vessels, directly to the tissue, or a combination thereof. In additional embodiments, an effective amount of isolated myogenic c-kit-BMCs is implanted in a patient in an encapsulating device (see, e.g., U.S. Pat. Nos. 9,132,226 and 8,425,928, the contents of each of which are incorporated herein by reference in their entirety).

In some embodiments of all aspects of the therapeutic methods described, an effective amount of isolated myogenic c-kit-BMCs is administered to a subject by systemic administration, such as intravenous administration.

The phrases “systemic administration,” “administered systemically”, “peripheral administration” and “administered peripherally” as used herein refer to the administration of population of myogenic c-kit-BMCs other than directly into the heart, such that it enters, instead, the subject's circulatory system.

In some embodiments of all aspects of the therapeutic methods described, one or more routes of administration are used in a subject to achieve distinct effects. For example, isolated myogenic c-kit-BMCs are administered to a subject by both direct injection and catheter-mediated routes for treating or repairing heart tissue. In such embodiments, different effective amounts of the isolated myogenic c-kit-BMCs can be used for each administration route.

In some embodiments of all aspects of the therapeutic methods described, the methods further comprise administration of one or more therapeutic agents, such as a drug or a molecule, that can enhance or potentiate the effects mediated by the administration of the isolated myogenic c-kit-BMCs, such as enhancing homing or engraftment of the myogenic c-kit-BMCs, increasing repair of cardiac cells, or increasing growth and regeneration of cardiac cells. The therapeutic agent can be a protein (such as an antibody or antigen-binding fragment), a peptide, a polynucleotide, an aptamer, a virus, a small molecule, a chemical compound, a cell, a drug, etc.

As defined herein, “vascular regeneration” refers to de novo formation of new blood vessels or the replacement of damaged blood vessels (e.g., capillaries) after injuries or traumas, as described herein, including but not limited to, heart disease. “Angiogenesis” is a term that can be used interchangeably to describe such phenomena.

In some embodiments of all aspects of the therapeutic methods described, the methods further comprise administration of myogenic c-kit-BMCs together with growth, differentiation, and angiogenesis agents or factors that are known in the art to stimulate cell growth, differentiation, and angiogenesis in the heart tissue. In some embodiments, any one of these factors can be delivered prior to or after administering the compositions described herein. Multiple subsequent delivery of any one of these factors can also occur to induce and/or enhance the engraftment, differentiation and/or angiogenesis. Suitable growth factors include but are not limited to ephrins (e.g., ephrin A or ephrin B), transforming growth factor-beta (TGFβ), vascular endothelial growth factor (VEGF), platelet derived growth factor (PDGF), angiopoietins, epidermal growth factor (EGF), bone morphogenic protein (BMP), basic fibroblast growth factor (bFGF), insulin and 3-isobutyl-1-methylxasthine (IBMX). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag, and these are hereby incorporated by reference in their entirety.

In one embodiment, the composition can include one or more bioactive agents to induce healing or regeneration of damaged heart tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Other bioactive agents can promote increased mitosis for cell growth and cell differentiation.

A great number of growth factors and differentiation factors are known in the art to stimulate cell growth and differentiation of stem cells and progenitor cells. Suitable growth factors and cytokines include any cytokines or growth factors capable of stimulating, maintaining, and/or mobilizing myogenic c-kit-BMCs and/or progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFβ, platelet derived growth factor (PDGF), angiopoietins (Ang), epidermal growth factor (EGF), bone morphogenic protein (BMP), fibroblast growth factor (FGF), hepatocyte growth factor (HGF), insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α. Other examples are described in Dijke et al., “Growth Factors for Wound Healing”, Bio/Technology, 7:793-798 (1989); Mulder G D, Haberer P A. Jeter K F, eds. Clinicians' Pocket Guide to Chronic Wound Repair. 4th ed. Springhouse, Pa.: Springhouse Corporation; 1998:85; Ziegler T. R, Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment of all aspects of the therapeutic methods described, the composition described is a suspension of myogenic c-kit-BMCs in a suitable physiologic carrier solution such as saline. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In certain embodiments of all aspects of the therapeutic methods described, the bioactive agent is a “pro-angiogenic factor,” which refers to factors that directly or indirectly promote new blood vessel formation in the heart. The pro-angiogenic factors include, but are not limited to ephrins (e.g., ephrin A or ephrin B), epidermal growth factor (EGF), E-cadherin, VEGF, angiogenin, angiopoietin-1, fibroblast growth factors: acidic (aFGF) and basic (bFGF), fibrinogen, fibronectin, heparanase, hepatocyte growth factor (HGF), angiopoietin, hypoxia-inducible factor-1 (HIF-1), insulin-like growth factor-1 (IGF-1), IGF, BP-3, platelet-derived growth factor (PDGF), VEGF-A, VEGF-C, pigment epithelium-derived factor (PEDF), vascular permeability factor (VPF), vitronection, leptin, trefoil peptides (TFFs), CYR61 (CCNI), NOV (CCN3), leptin, midkine, placental growth factor platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), c-Myc, granulocyte colony-stimulating factor (G-CSF), stromal derived factor 1 (SDF-1), scatter factor (SF), osteopontin, stem cell factor (SCF), matrix metalloproteinases (MMPs), thrombospondin-1 (TSP-1), pleitrophin, proliferin, follistatin, placental growth factor (PIGF), midkine, platelet-derived growth factor-BB (PDGF), and fractalkine, and inflammatory cytokines and chemokines that are inducers of angiogenesis and increased vascularity, e.g., interleukin-3 (IL-3), interleukin-8 (IL-8), CCL2 (MCP-1), interleukin-8 (L-8) and CCL5 (RANTES).

Suitable dosage of one or more therapeutic agents in the compositions described herein can include a concentration of about 0.1 to about 500 ng/ml, about 10 to about 500 ng/ml, about 20 to about 500 ng/ml, about 30 to about 500 ng/ml, about 50 to about 500 ng/ml, or about 80 ng/ml to about 500 ng/ml. In some embodiments, the suitable dosage of one or more therapeutic agents is about 10, about 25, about 45, about 60, about 75, about 100, about 125, about 150, about 175, about 200, about 225, about 250, about 275, about 300, about 325, about 350, about 375, about 400, about 425, about 450, about 475, or about 500 ng/ml. In other embodiments, suitable dosage of one or more therapeutic agents is about 0.6, about 0.7, about 0.8, about 0.9, about 1.0, about 1.5, or about 2.0 μg/ml.

In some embodiments of all aspects of the therapeutic methods described, the standard therapeutic agents for heart disease are those that have been described in detail, see, e.g., Harrison's Principles of Internal Medicine, 15th edition, 2001, E. Braunwald, et al., editors, McGraw-Hill, New York, N.Y., ISBN 0-07-007272-8, especially chapters 252-265 at pages 1456-1526; Physicians Desk Reference 54th edition, 2000, pages 303-3251, ISBN 1-56363-330-2, Medical Economics Co., Inc., Montvale, N.J. Treatment of any heart disease or disorder can be accomplished using the treatment regimens described herein. For chronic conditions, intermittent dosing can be used to reduce the frequency of treatment. Intermittent dosing protocols are as described herein.

For the clinical use of the methods described herein, isolated populations of myogenic c-kit-BMCs described herein can be administered along with any pharmaceutically acceptable compound, material, carrier or composition which results in an effective treatment in the subject. Thus, a pharmaceutical formulation for use in the methods described herein can contain an isolated myogenic c-kit-BMCs in combination with one or more pharmaceutically acceptable ingredients.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent, media (e.g., stem cell media), encapsulating material, manufacturing aid (e.g., lubricant, talc magnesium, calcium or zinc stearate, or steric acid), or solvent encapsulating material, involved in maintaining the activity of, carrying, or transporting the isolated myogenic c-kit-BMCs from one organ, or portion of the body, to another organ, or portion of the body.

Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) phosphate buffered solutions; (3) pyrogen-free water; (4) isotonic saline; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) cellulose, and its derivatives, such as sodium carboxymethyl cellulose, methylcellulose, ethyl cellulose, microcrystalline cellulose and cellulose acetate; (17) powdered tragacanth; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C2-C12 alcohols, such as ethanol; (25) starches, such as corn starch and potato starch; and (26) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In one embodiment, the invention provides a method of producing myogenic c-kit positive bone marrow cells (c-kit-BMCs), comprising: (a) isolating c-kit positive bone marrow cells from bone marrow; (b) selecting myogenic c-kit positive bone marrow cells (c-kit-BMCs) from step (a); and (c) culturing and expanding the c-kit-BMCs of step (b), thereby producing c-kit-BMCs. The selecting step may comprise selecting c-kit-BMCs having enhanced expression of RYR3, OSM, Jag1, Hey2 and Smyd3. A population of myogenic c-kit-BMCs may be substantially enriched for c-kit-BMCs that have enhanced expression of RYR3, OSM, Jag1, Hey2 and Smyd3. Any suitable technique for the sorting of cells (e.g., FACS) may be used for the selecting step.

The term “substantially enriched,” with respect to a particular cell population, refers to a population of cells that is at least about 50%, 75%, at least about 80%, at least about 85%, at least about 90%, at least about 95%, at least about 98%, or at least about 99% pure, with respect to the cells making up a total cell population. In other words, the terms “substantially enriched” or “essentially purified”, with regard to a population of myogenic c-kit-BMCs isolated for use in the methods disclosed herein, refers to a population of myogenic c-kit-BMCs that contain fewer than about 30%, 25%, fewer than about 20%, fewer than about 15%, fewer than about 10%, fewer than about 90%, fewer than about 8%, fewer than about 70%, fewer than about 6%, fewer than about 5%, fewer than about 4%, fewer than about 3%, fewer than about 2%, fewer than about 1%, or less than 1%, of cells that are not myogenic c-kit-BMCs, as defined by the terms herein. Some embodiments of these aspects further encompass methods to expand a population of substantially pure or enriched myogenic c-kit-BMCs, wherein the expanded population of myogenic c-kit-BMCs is also a substantially pure or enriched population of myogenic c-kit-BMCs.

In some embodiments, the isolated or substantially enriched myogenic c-kit-BMC populations obtained by the methods disclosed herein are later administered to a second subject, or re-introduced into the subject from which the cell population was originally isolated (e.g., allogeneic transplantation vs. autologous administration).

Unless otherwise defined, all technical and scientific terms used herein have the same meaning as commonly understood by one of ordinary skill in the art to which this invention belongs. Certain terms employed herein, in the specification, examples and claims are collected here.

As used herein, “in vivo” (Latin for “within the living”) refers to those methods using a whole, living organism, such as a human subject. As used herein, “ex vivo” (Latin: out of the living) refers to those methods that are performed outside the body of a subject, and refers to those procedures in which an organ, cells, or tissue are taken from a living subject for a procedure, e.g., isolating a specific population of c-kit-BMCs from heart tissue obtained from a donor subject, and then administering the isolated specific population of c-kit-BMCs to a recipient subject. As used herein, “in vitro” refers to those methods performed outside of a subject, such as an in vitro cell culture experiment. For example, a specific population of c-kit-BMCs can be cultured in vitro to expand or increase the number of specific c-kit-BMCs, or to direct differentiation of the c-kit-BMCs to a specific lineage or cell type, e.g., cardiomyocytes, endothelial cells, fibroblasts, coronary vessels and/or cells of mesodermal origin prior to being used or administered according to the methods described herein.

The term “pluripotent” as used herein refers to a cell with the capacity, under different conditions, to commit to one or more specific cell type lineage and differentiate to more than one differentiated cell type of the committed lineage, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells) also have the characteristic of the capacity of extended passaging without loss of growth potential, relative to primary cell parents, which generally have capacity for only a limited number of divisions in culture.

The term “progenitor” cell are used herein refers to cells that have a cellular phenotype that is more primitive (i.e., is at an earlier step along a developmental pathway or progression than is a fully differentiated or terminally differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Progenitor cells give rise to precursor cells of specific determinate lineage, for example, certain cardiac progenitor cells divide to give cardiac cell lineage precursor cells. These precursor cells divide and give rise to many cells that terminally differentiate to, for example, cardiomyocytes.

The term “precursor” cell is used herein refers to a cell that has a cellular phenotype that is more primitive than a terminally differentiated cell but is less primitive than a stem cell or progenitor cell that is along its same developmental pathway. A “precursor” cell is typically progeny cells of a “progenitor” cell which are some of the daughters of “stem cells”. One of the daughters in a typical asymmetrical cell division assumes the role of the stem cell.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that the cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” is used to refer to any multipotent stem cell derived from non-embryonic tissue, including juvenile and adult tissue. In some embodiments, adult stem cells can be of non-fetal origin.

In the context of cell ontogeny, the adjective “differentiated” or “differentiating” is a relative term meaning a “differentiated cell” is a cell that has progressed further down the developmental pathway than the cell it is being compared with. Thus, stem cells can differentiate to lineage-restricted precursor cells (such as a cardiac stem cell), which in turn can differentiate into other types of precursor cells further down the pathway (such as an exocrine or endocrine precursor), and then to an end-stage differentiated cell, which plays a characteristic role in a certain tissue type, and may or may not retain the capacity to proliferate further.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a myogenic c-kit-BMC) in a cellular differentiation process.

As used herein, the term “somatic cell” refers to any cell forming the body of an organism, as opposed to germ line cells. In mammals, germ line cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

The term “phenotype” refers to one or a number of total biological characteristics that define the cell or organism under a particular set of environmental conditions and factors, regardless of the actual genotype. For example, the expression of cell surface markers in a cell. The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The terms “renewal” or “self-renewal” or “proliferation” are used interchangeably herein, are used to refer to the ability of stem cells to renew themselves by dividing into the same non-specialized cell type over long periods, and/or many months to years.

In some instances, “proliferation” refers to the expansion of cells by the repeated division of single cells into two identical daughter cells.

The term “lineages” is used herein describes a cell with a common ancestry or cells with a common developmental fate.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” typically means a decrease by at least about 5%-10% as compared to a reference level, for example a decrease by at least about 20%, or at least about 30%, or at least about 40%, or at least about 50%, or at least about 60%, or at least about 70%, or at least about 80%, or at least about 90% decrease (i.e., absent level as compared to a reference sample), or any decrease between 10-90% as compared to a reference level. In the context of treatment or prevention, the reference level is a symptom level of a subject in the absence of administering a population of myogenic c-kit-BMCs.

The terms “increased”, “increase” or “enhance” are all used herein to generally mean an increase by a statically significant amount; for the avoidance of any doubt, the terms “increased”, “increase” or “enhance” means an increase of at least 10% as compared to a reference level, for example an increase of at least about 20%, or at least about 30%, or at least about 40%, or at least about 50%, or at least about 60%, or at least about 7%, or at least about 80%, or at least about 90% increase or more, or any increase between 10-90% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of myogenic c-kit-BMCs' expansion in vitro, the reference level is the initial number of myogenic c-kit-BMCs isolated from a sample obtained from a subject.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2nd ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R Ian Freshney, Publisher: Wiley-Liss; 5th edition (2005) and Animal Cell Culture Methods (Methods in Cell Biology, Vol. 57, Jennie P. Mather and David Barnes editors, Academic Press, 1st edition, 1998) which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.”

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The invention will be further clarified by the following examples, which are intended to be purely exemplary and in no way limiting.

### EXAMPLES

### Example 1: Methods

Briefly, bone marrow was harvested from the femurs and tibias of C57131/6 mice at 2 months of age. Cells were incubated with CD117-microbeads, enriched by MACS and infected with a GFP-lentivirus. Subsequently, FACS-sorted GFP-labeled c-kit-BMCs were injected in infarcted mice. Two weeks later, hearts were enzymatically digested to obtain cardiomyocytes, endothelial cells (ECs), fibroblasts and c-kit-BMCs. Genomic DNA was extracted and the sites of viral integration were identified by PCR Additionally, c-kit-BMCs were infected with three lentiviruses carrying mCherry, YFP or CFP and delivered to infarcted hearts; 4-7 and 14-21 days later, hearts were formalin-fixed and newly formed structures were recognized by immunolabeling.

In a separate set of experiments, GFP-positive c-kit-BMCs were FACS-sorted and seeded at limiting dilution for single cell-derived clone formation. Clonal cells were injected in infarcted mice, and, 21 days later, the site of viral integration was determined in regenerated cardiomyocytes. Following the identification of clonal c-kit-BMCs able and unable to form cardiomyocytes, BMCs were subjected to RNA sequencing to define the molecular signature of these two classes of BMCs.

Data are presented as mean±SD. Differential expression of genes was computed by Cufflinks (version 2.0.2) with iGenome's UCSC HG19 annotation. P<0.05 was considered significant.

1.1 Detection of Sites of Viral Integration in Cardiac Cells

a) Culture and lentiviral infection of c-kit-BMCs. The bone marrow was harvested from the femurs and tibias of C57Bl/6 mice at 2 months of age.^{1,2} Lysis of erythrocytes was obtained by incubating bone marrow cells (BMCs) with BO Pharm Lyse™ (Beckton Dickinson) for 15-20 min at room temperature. Bone marrow mononuclear cells were washed out with PBS containing 0.5% bovine serum albumin (BSA) and 2 mM EDTA (Gibco). Cells were re-suspended in washing buffer and incubated with CD117-microbeads (Miltenyi) for 15 min at 4° C. c-kit-BMCs were enriched by MACS and plated in non-coated dishes for 2 days. Cells were cultured with Iscove's Modified Dulbecco's Medium (IMDM, Invitrogen), supplemented with thrombopoietin (TPO, 20 ng/ml), interleukin-3 (IL-3, 20 ng/ml), interleukin-6 (IL-6, 40 ng/ml), Fms-related tyrosine kinase 3 ligand (Flt3, 10 ng/ml), stem cell factor (SCF, 50 ng/ml), and 10% fetal bovine serum (FBS) in the presence of penicillin and streptomycin.³ GFP-lentiviral supernatant was added to retronectin-coated (Takara) dishes. Floating c-kit-BMCs were then transferred, 2×10⁵ cells/dish, and expanded for 3 days.

b) Myocardial infarction and transplantation of GFP-labeled c-kit-BMCs. All protocols were approved by the Institutional Animal Care and Use Committee (IACUC) of the Brigham and Women's Hospital. Animals received humane care in compliance with the Guide for the Care and Use of laboratory Animals as described by the Institute of Laboratory Animal Research Resources, Commission on life Sciences, National Research Council. Myocardial infarction was induced in anesthetized (isoflurane 1.5%) female C57Bl/6 mice at 3 months of age as previously described. Shortly after coronary artery ligation, FACS-sorted GFP-labeled c-kit-BMCs, 1×10⁵ per heart, were injected in four different sites of the region bordering the infarct.^{1,2,4} Animals were sacrificed two weeks later.

c) Enzymatic dissociation and isolation of cardiac cells. At sacrifice, hearts were enzymatically digested with protease and collagenase type II (Worthington) to obtain a single cell suspension.2.5.6 Hearts were excised and placed on a stainless steel cannula for retrograde perfusion through the aorta. The solutions were supplements of modified commercial MEM Joklik (Sigma). HEPES/MEM contained 117 mM NaCl, 5.7 mM KCl, 4.4 mM NaHCO₃, 1.5 mM KH₂PO₄, 17 mM MgCl₂, 21.1 mM HEPES, 11.7 mM glucose, amino acids, and vitamins, 2 mM L-glutamine, 10 mM taurine, and 21 mU/ml insulin and adjusted to pH 7.2 with NaOH. Resuspension medium was HEPES/MEM supplemented with 0.5% BSA, 0.3 mM calcium chloride, and 10 mM taurine. The cell isolation procedure consisted of four main steps. 1) Calcium-free perfusion: blood washout and collagenase type II-perfusion of the heart was carried out at 34° C. with HEPES/MEM gassed with 85% 02 and 15% N₂. 2) Mechanical tissue dissociation: after the heart was removed from the cannula, the collagenase-perfused myocardium was minced and subsequently shaken in resuspension medium containing collagenase. 3) Myocyte separation: cells were centrifuged at 30 g for 3 min. This procedure was repeated four to five times. Myocytes were recovered from the pellet and washed, and the supernatant was collected. 4) Separation of small cardiac cells:5 cells were obtained from the supernatant and sorted by FACS with antibodies recognizing c-kit, CD31 and Thy1.2. ECs were positive for CD31 and negative for Thy1.2 and c-kit; fibroblasts were positive for Thy1.2 and negative for CD31 and c-kit; and BMCs were positive for c-kit only. The purity of myocytes, ECs, fibroblasts and c-kit-BMCs was documented by immunolabeling and fluorescent microscopy and RT-PCR

d) Purity of the isolated populations of cardiac cells. Isolated cardiomyocytes and FACS-sorted ECs and fibroblasts were fixed in suspension with 4% paraformaldehyde (PFA). Aliquots of cells were deposited on a slide and labeled, respectively, with antibodies recognizing α-sarcomeric actin (α-SA, Sigma), von Willebrand factor (vWF, Abeam), and procollagen (Pro-Col, Abeam). Nuclei were stained by DAPI. The fraction of cells positive for lineage markers was then determined.

For qRT-PCR, total RNA was isolated from myocytes, FACS-sorted c-kit-BMCs, ECs, and fibroblasts using an RNeasy mini kit (Qiagen). Total RNA was converted to complementary DNA (cDNA) using High Capacity cDNA synthesis kit (Applied Biosystems). qRT-PCR was performed on 7300 Real Time PCR System (Applied Biosystems) using 1/20th of the cDNA per reaction. Primers were designed from available mouse sequences using the primer analysis software Vector NTI (Invitrogen). Transcripts of α-cardiac myosin heavy chain (Myh6), CD31, collagen type III, α-1 (Col3a1), c-kit and the housekeeping gene β-2 microglobulin (B2M) were measured. Mouse myocardium was used as control. The PCR-reaction included 1 μl template cDNA, 500 nM forward and reverse-primers in a total volume of 20 μl. Cycling conditions were as follows: 95° C. for 10 min followed by 35 cycles of amplification (95° C. denaturation for 15 sec, and 60° C. combined annealing/extension for 1 min). Primers were as follows:

Myh6-Forward:
(SEQ ID NO: 1)
5′-ACC AAC CTG TCC AAG TTC CG-3′
Myh6-Reverse:
(SEQ ID NO: 2)
5′-TAT TGG CCA CAG CGA GGG TC-3′
CD31-Forward:
(SEQ ID NO: 3)
5′-AGC TGC TCC ACT TCT GAA CTC-3′
CD31-Reverse:
(SEQ ID NO: 4)
5′-TCA AGG GAG GAC ACT TCC AC-3′
Col3a1-Forward:
(SEQ ID NO: 5)
5′-GGT GAC AGA GGA GAA ACT GG-3′
Col3a1-Reverse:
(SEQ ID NO: 6)
5′-ATG TGG TCC AAC TGG TCC TC-3′
B2M-Forward:
(SEQ ID NO: 7)
5′-CTC GGT GAC CCT GGT CTT TC-3′
B2M-Reverse:
(SEQ ID NO: 8)
5′-TTC AGT ATG TTC GGC TTC CC-3′

RT-PCR products were run on 2% agarose/1×TAE gel and bands of distinct molecular weight were identified.

e) Identification of proviral integrants in the mouse genome. Each integration site corresponds to a distinctive genomic sequence, which was detected on the assumption that a restriction enzyme (RE) cleavage site was present at a reasonable distance (20-800 bp) from long terminal repeats (LTRs) flanking the viral genome. Following the cleavage of the genomic DNA with the RE, DNA products were self-ligated to produce circularized DNA.^{5,7,9} Different primers and distinct RE were employed to optimize the methodology of detection of the viral integration site. This step created a genomic sequence of variable length due to the random location of the RE site within the lentiviral flanking region. Since the unknown lentiviral flanking region was entrapped between two known sequences, it was possible to amplify the viral integration site by PCR.

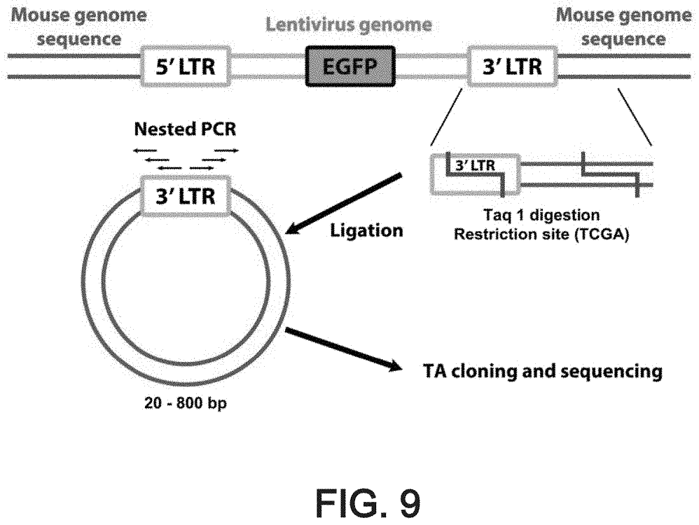

Genomic DNA was extracted from cardiomyocytes, ECs, fibroblasts and c-kit-BMCs with QIAamp DNA Mini Kit (QIAGEN). The extracted DNA was digested with Taq I (New England Biolabs) for 2 h at 65° C. The enzyme was heat-inactivated at 80° C. for 25 min. Aliquots of samples were run on agarose gel to confirm digestion. To circularize DNA fragments, samples were incubated with 10 μl Quick T4 DNA Ligase (New England Biolabs) in a total reaction volume of 200 μl and kept at room temperature overnight. Phenol/chloroform and chloroform extractions were then performed. After 2-propanol precipitation, DNA was re-linearized with Hind III (10 U). The protocol utilized for the recognition of the integrated provirus corresponds to an inverse PCR, which is the most sensitive strategy for the amplification of unknown DNA sequences that flank a region of known sequence.⁷ The primers are oriented in the reverse direction of the usual orientation and the template is a restriction fragment that has been ligated to be self-circularized. One round of PCR and two additional nested PCR were performed utilizing AccuPrime Pfx SuperMix (Invitrogen). At each PCR step, samples were diluted 1:2,500. The PCR primers employed in the first (1st) and second (2nd) amplification round were designed in the region of LTR which is commonly located at the 5′- and 3′-side of the lentiviral genome. The PCR primers employed in the third round (3rd) were specific for the 3′-side of the site of integration. In all cases, primers were oriented in the opposite direction (FIG. 9).

First Round PCR:
eGFP-X:
(SEQ ID NO: 9)
GGTTCCCTAGTTAGCCAGAGAGC
(23 nt)
eGFP-Y:
(SEQ ID NO: 10)
GAGTGCTTCAAGTAGTGTGTGC
(22 nt)
95º C. for 5 min;
40 cycles of 95º C. for 15 sec,
55º C. for 30 sec, 68º C.
for 70 sec; 68º C. for 2 min.
Second Round PCR:
eGFP-M:
(SEQ ID NO: 11)
AGCAGATCTTGTCTTCGTTGGGAGTG
(26 nt)
eGFP-Z:
(SEQ ID NO: 12)
CCGTCTGTTGTGTGACTCTGGTAA
(24 nt)
same cycling condition as above
but with 25 cycles.
Third Round PCR:
eGFP-F:
(SEQ ID NO: 13)
5′-CATTGGTCTTAAAGGTACCGAGCTCG-3′
eGFP-L:
(SEQ ID NO: 14)
5′-GATCCCTCAGACCCTTTTAGTCAGTG-3′
same cycling condition as the second round.

Taq polymerase-amplified PCR products were inserted into the plasmid vector pCR4-TOPO using the TOPO TA Cloning Kit (Invitrogen). Subsequently, chemically competent TOP10 E. coli cells were transformed with the vector carrying the PCR products. The transformation mixture was spread on agar plates and incubated overnight at 37° C. Ten to twenty colonies from each plate were expanded in 10 ml LB medium containing ampicillin. The amplified constructs were extracted with the QIAGEN Plasmid Purification Mini-Kit, digested with EcoRI, and run on agarose gel. Bands of different molecular weight were identified. DNA sequencing was performed to verify the presence of viral integration sites.

1.2 Red, Green and Blue (RGB) Marking of c-Kit-BMCs

a) Culture and lentiviral infection of c-kit-BMCs. c-kit-BMCs were cultured (see above) and concurrently infected with three lentiviral vectors carrying distinct fluorochromes.¹⁰⁻¹² The following viruses were employed: 1) EX-mChER-Lv105-vector with mCherry for pReceiver-Lv105, which corresponds to an HIV-based lenti-vector with a CMV promoter and puromycin selection marker; 2) EX-eYFP-Lv102-vector with enhanced yellow fluorescent protein (eYFP) for pReceiver-Lv 102, which corresponds to an HIV-based lenti-vector with a CMV promoter, N-FLAG tag and puromycin selection marker; and 3) EX-eCFP-Lv107-vector with enhanced cyan fluorescent protein (eCFP) for pReceiver-Lv107, which corresponds to an HIV-based lenti-vector with a CMV promoter, N-Myc tag.

b) In vitro detection of fluorescent markers. Native fluorescence of mCherry, eYFP and eCFP in c-kit-BMCs was established by epifluorescence microscopy. The presence of the three primary colors and their combinations was detected in the majority of c-kit-BMCs. The quantitative analysis of the proportion of c-kit-BMCs infected by one, two or three vectors was performed by FACS.

c) Myocardial infarction and transplantation of RGB-labeled c-kit-BMCs. Myocardial infarction was induced as described above. Acutely after coronary artery ligation, 1×10⁵ c-kit-BMCs infected with the three lentiviruses carrying mCherry, YFP or CFP were injected at 4 sites in the region bordering the infarct.^{1,2,4,5,13} Animals were sacrificed 4-7 and 14-21 days later. Briefly, the abdominal aorta was cannulated with a polyethylene catheter filled with heparin-sodium injection solution (1,000 units/ml). In rapid succession, the heart was arrested in diastole by injection of cadmium chloride (100 mM), and perfusion with phosphate buffer was conducted for ˜3 min. The thorax was then opened, and the right atrium was cut to allow drainage of blood and perfusate. The heart was fixed by perfusion with 10% phosphate-buffered formalin. After fixation, the heart was dissected, and sections from the base and mid-portion of the left ventricle were examined.^{1,2,4,5,13} Immunolabeling was performed with: mouse monoclonal mCherry antibody (1051; Abcam) for the detection of mCherry; rabbit polyclonal DDDDK (SEQ ID NO:15) tag antibody (Abcam) for the detection of the N-FLAG tag in the eYFP lentivirus; and chicken polyclonal Myc tag antibody (Abcam) for the detection of the N-Myc tag in the eCFP lentivirus.

c) At 14 days after infarction, LV hemodynamics loops were obtained in untreated (n=11) and cell treated (n=8) mice. The parameters were obtained in the closed-chest preparation with a MPVS-400 system for small animals (Millar Instruments) equipped with a PVR-1045 catheter.^{14,15} Mice were intubated and ventilated (MiniVent Type 845; Hugo Sachs Elektronik-Harvard Apparatus, GmbH, March, Germany) with isoflurane anesthesia (isoflurane, 1.5%); the right carotid artery was exposed and the pressure transducer was inserted and advanced in the LV cavity. Data were acquired with LabChart (ADInstruments) software.

1.3 Clonal Assay for the Identification of Myogenic c-Kit-BMCs

a) Preparation of c-kit-BMC clones and in vivo transplantation. Freshly isolated c-kit-BMCs were infected with a lentivirus carrying GFP. Subsequently, c-kit-positive GFP-positive BMCs were FACS-sorted and seeded at limiting dilution in Methocult-coated wells (3×10³ per well). Over a period of 10 days, small colonies derived from individual BMCs were observed. Cells were further expanded and the expression of c-kit and GFP was determined; 15 clones were employed for in vivo assays and DNA and RNA extraction. A total of 1×10⁵ cells, i.e., 2×10⁴ from each of 5 clones, were injected in the border zone of acutely infarcted mice, and the animals were sacrificed 21 days later for the detection of the site of viral integration in regenerated cardiomyocytes. Cardiomyocytes were collected by enzymatic digestion as describe above. Additionally, the site of integration in c-kit-positive GFP-positive BMCs formed in each clone was determined to establish the lineage relationship between specific clonal cells and the cardiomyocyte progeny. Following the identification of clonal c-kit-BMCs able and unable to form cardiomyocytes, BMCs were subjected to RNA sequencing to define the molecular signature of these two classes of BMCs.¹⁶

b) RNA-sequencing. Clonal myogenic c-kit-BMCs, clonal non-myogenic c-kit-BMCs and freshly isolated FACS-sorted c-kit-BMCs were utilized in this assay. RNA was isolated using an RN easy mini kit (Qiagen), and 100 ng of total RNA was converted to complementary DNA (cDNA) and amplified using NuGEN V2 RNA-Seq kit (NuGEN). cDNA was sonicated to an average fragment size of 300 bp and Illumina sequencing adapters were ligated to 500 ng of cDNA using NEBNext mRNA Library Prep Reagent Set for Illumina (New England Biolabs). Sequencing was performed using Illumina's HiSeq2000 platform using paired in reads at an average length of 100 bp. The alignment to human hg19 assembly was done by Tophat (version 2.0.5).

1.4 Statistical Analysis

Data are presented as mean±SD. Differential expression of genes was computed by Cufflinks (version 2.0.2) with iGenome's UCSC HG19 annotation. P<0.05 was considered significant. For the hemodynamic data the two tailed unpaired Student's t-test was applied.

### Example 2: Results

2.1 Phenotype and Viral Gene Tagging of c-Kit-BMCs

Mouse c-kit-BMCs were enriched with immunomagnetic beads and cultured in non-coated dishes for 2 days in the presence of growth factors to increase the fraction of cycling cells and their sensitivity to lentiviral infection. Floating cells were transferred to RetroNectin-coated dishes and cultured for an additional 3 days in the presence of viral particles carrying GFP to obtain fluorescently labeled cells. To determine whether c-kit-BMCs transdifferentiate and form a cardiomyocyte progeny in vivo, myocardial infarction was induced by coronary ligation in syngeneic mice (n=8). Shortly after coronary occlusion, 1×10⁵ GFP-positive c-kit-BMCs were injected in four different sites of the region bordering the infarct. All animals were treated with GFP-positive c-kit-BMCs collected from the same preparation to ensure that cells with identical viral integration sites were delivered to the myocardium of the 8 infarcted mice studied. Two weeks after surgery and cell implantation, the infarcted heart was enzymatically dissociated with collagenase to obtain a single cell suspension.

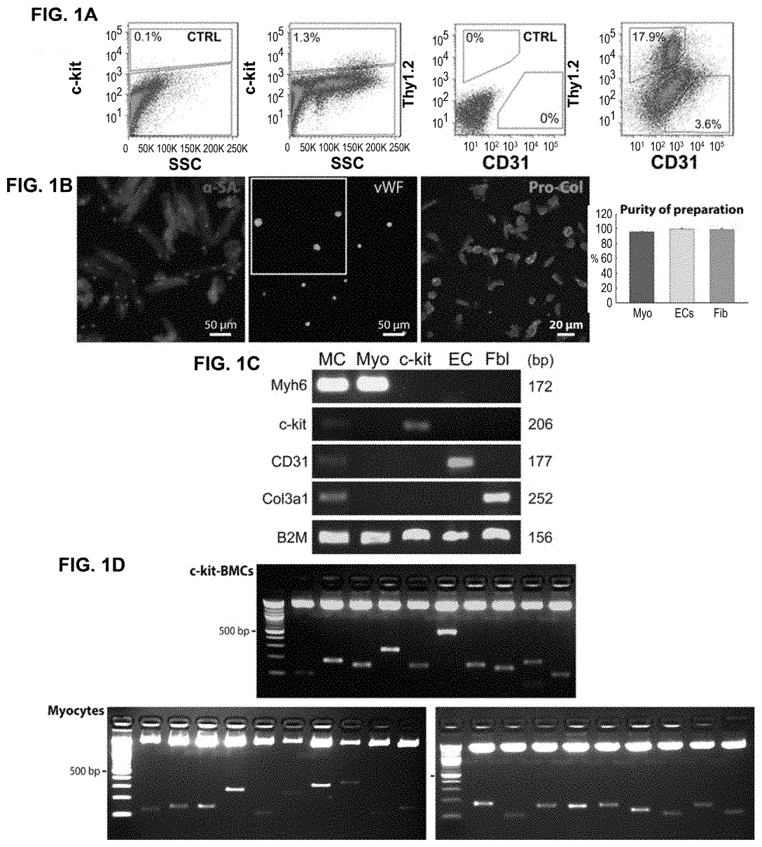

Myocytes were purified by differential centrifugation, while ECs, fibroblasts and c-kit-positive cells were sorted by flow-cytometer based on the expression of CD31, Thy1.2 and c-kit, respectively. ECs were positive for CD31, and negative for c-kit and Thy1.2, fibroblasts were positive for Thy1.2, and negative for c-kit and CD31, and c-kit-positive cells expressed this epitope but were negative for CD31 and Thy1.2 (FIG. 1A). Aliquots from each cell sample were fixed in paraformaldehyde and their purity was determined by immunolabeling and confocal microscopy. In all cases, the level of contamination from other cardiac cells was negligible (FIG. 1B). Vascular smooth muscle cells were not included in this analysis; they represent a minimal fraction of the cardiac cell populations and cannot be acquired in reasonable quantity.

RT-PCR was employed to confirm that transcripts for α-myosin heavy chain (Myh6), CD31 and procollagen (Col3a1) were restricted, respectively, to myocytes, ECs and fibroblasts (FIG. 1C). Moreover, the expression of c-kit in these three differentiated cell populations was evaluated to assess the presence of contaminant c-kit-positive cells; c-kit mRNA was not found in myocyte, EC and fibroblast preparations (FIG. 1C). Thus, these protocols are satisfactory for the analysis of the site of viral integration in the genome of each cardiac cell population.

2.2 Sites of Viral Integration in c-Kit-BMCs, Cardiomyocytes and ECs

Myocytes, ECs, fibroblasts and c-kit-positive cells isolated from infarcted hearts treated with c-kit-BMCs were analyzed for the detection of proviral integrants in the mouse genome. Each insertion site corresponded to a specific genomic sequence, which was detected on the assumption that the cleavage site of the Taq I restriction enzyme was present at a distance of 20-800 bp from long terminal repeats (L TRs) flanking the viral genome (FIG. 9). Thus, a genomic sequence of variable length was created based on the random location of the Taq I site within the lentiviral flanking region. The viral integration site was amplified by nested PCR, since the unknown lentiviral flanking region was entrapped between two known sequences. Circularized DNA was linearized by digestion with Hind III to enhance the sensitivity of this protocol. The PCR products were subjected to TA cloning and transduced in E. coli. From each preparation, 10-20 developed bacterial colonies were collected for myocytes, ECs, fibroblasts and c-kit-positive cells in each animal and grown for an additional 16 hours. DNA was digested with EcoR 1 and run on agarose gel; multiple bands of distinct molecular weights were identified (FIG. 1D).

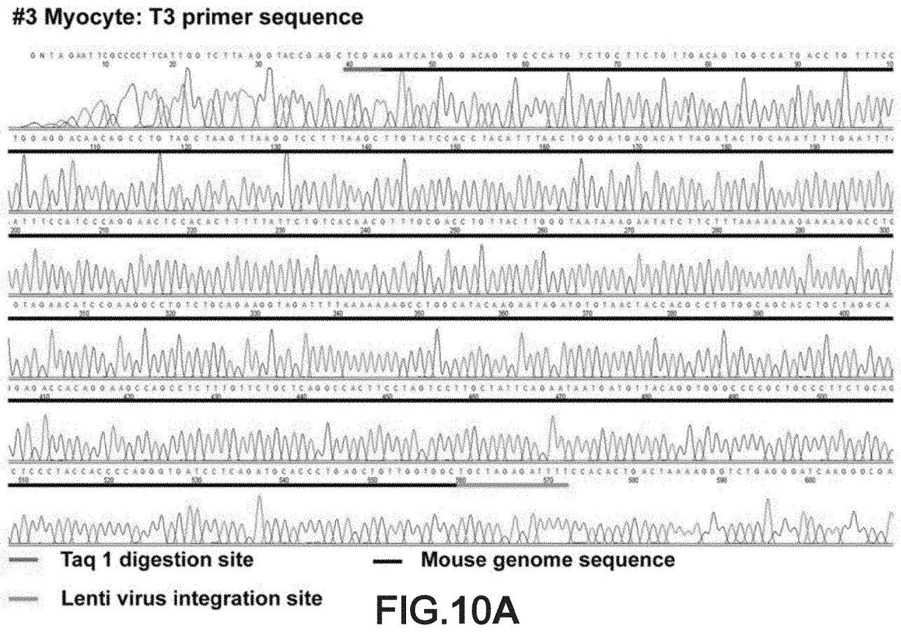

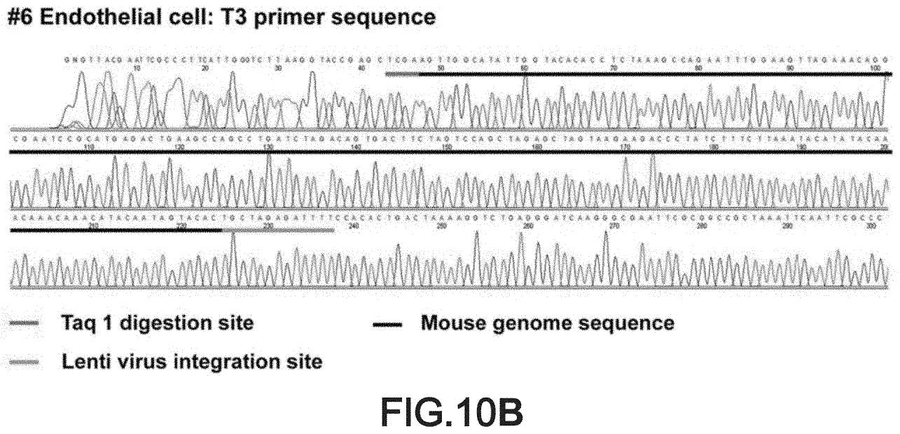

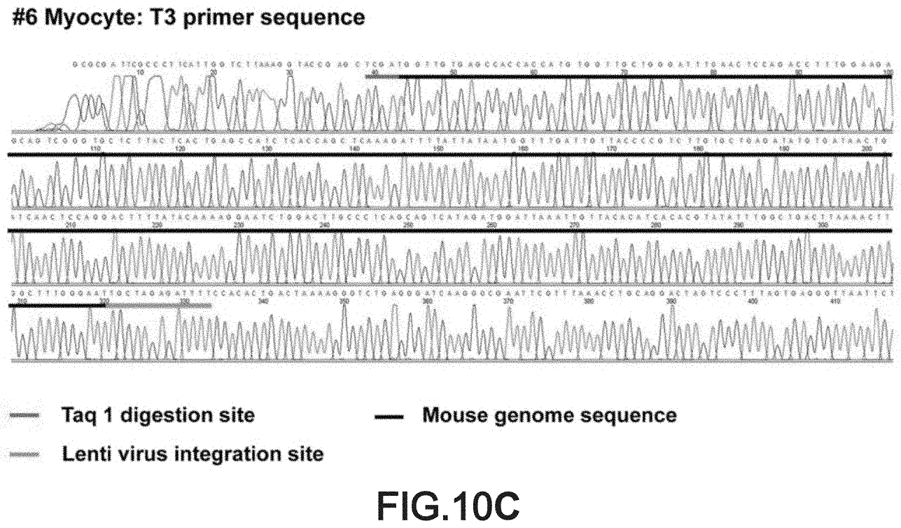

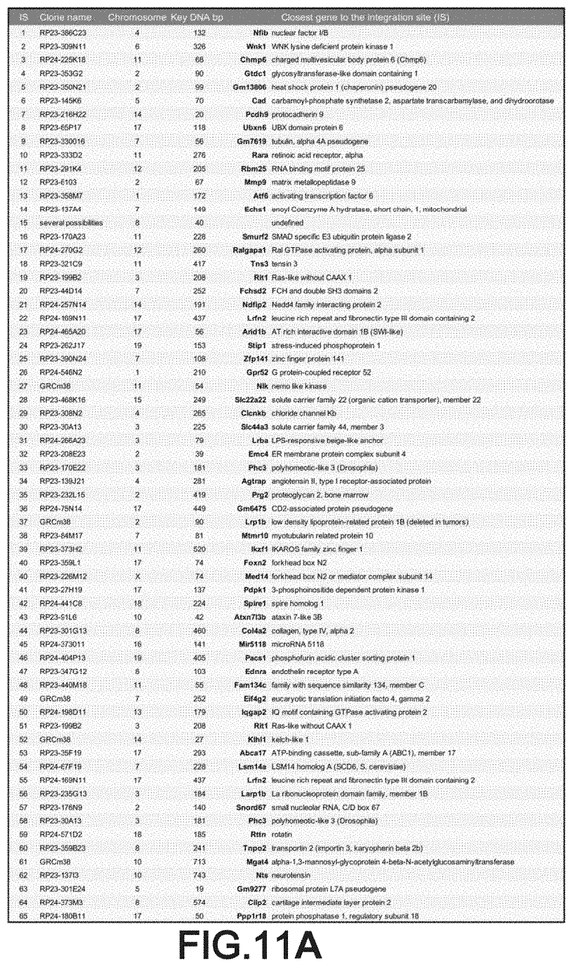

By sequence analysis, the purified DNA contained the viral and mouse genome, and thereby corresponded to proviral integrant sites (FIG. 10). A total of 111 clones were identified in 7 of 8 independent experiments, and 65 of the 111 clones reflected different sites of integration (FIGS. 11A and 11B). Of the 65 viral clones, 13 derived from myogenic mother c-kit-BMCs, 18 from vasculogenic mother c-kit-BMCs, 10 from fibrogenic mother c-kit-BMCs and 12 from self-renewing undifferentiated mother c-kit-BMCs. In 12 cases, common integration sites were detected in c-kit-BMCs, myocytes, ECs and fibroblasts in various combinations documenting a lineage relationship between the delivered c-kit-BMCs and the diverse cardiac cell phenotypes (FIGS. 11A and 11B). Thus, clonal expansion and lineage commitment of individual c-kit-BMCs occur in vivo, supporting the notion that c-kit-BMCs transdifferentiate and repair the infarcted heart.

2.3 Multicolor Clonal Tracking of c-Kit-BMCs and their Progeny

Three lentiviral vectors carrying, respectively, mCherry (red), YFP (yellow) and CFP (cyan) fluorescent protein were employed to infect c-kit-BMCs. Each color and their combination were evaluated in vitro in c-kit-BMCs with the expectation that a similar pattern of colors could be detected later in tissue sections by immunolabeling and confocal microscopy. Structures sharing common labeling were anticipated to represent the progeny derived from clonal expansion and differentiation of individual c-kit-BMCs.

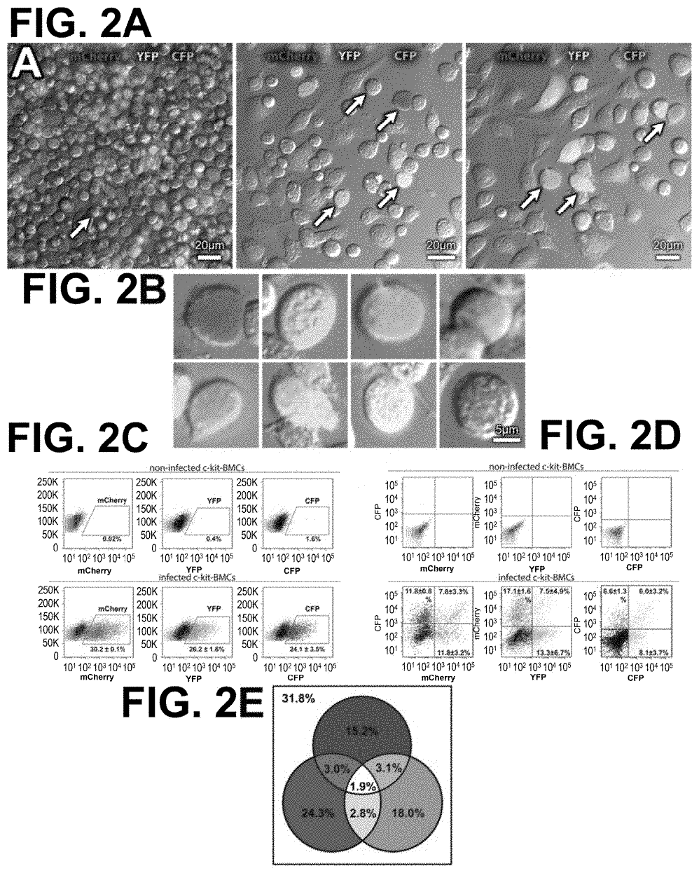

The fluorescent signals in c-kit-BMCs were detected in vitro by native red, yellow and cyan fluorescence (FIGS. 2A and 2B). These qualitative observations were complemented with a flow cytometry analysis to evaluate quantitatively distinctly labeled cell clusters (FIGS. 2C and 2D). Based on the additive color theory, we assigned the 3 primary colors, i.e., red, green and blue, to mCherry, YFP and CFP, respectively. These basic colors give rise to secondary colors formed by the mixture of red, green and blue. If red and green are mixed, bright yellow is generated, while a mixture of red and blue results in violet, and a mixture of blue and green results in turquoise.⁶

Eight separate cell categories were identified: they included c-kit-BMCs transduced with only one of each of the 3 viral vectors; these cells showed red fluorescence in 24.3% of the cases, green in 18%, and blue in 15.2%. Three more classes of cells showed the combination of red and green, i.e., yellow: 2.8%; red and blue, i.e., violet: 3.0%; and green and blue, i.e., turquoise: 3.1%. One cell category was labeled by red, green and blue, i.e., white: 1.9%; and one was unlabeled, 31.8% (FIG. 2E).

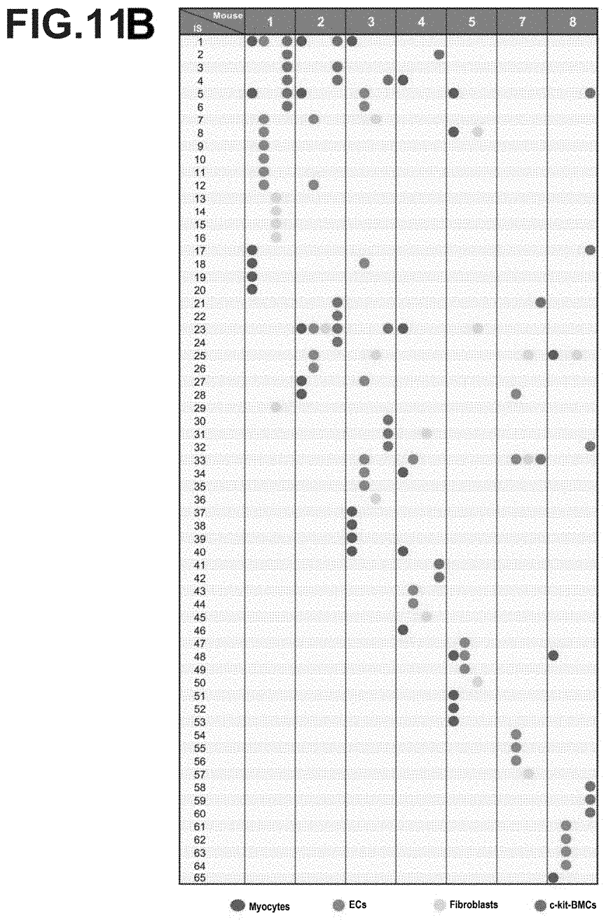

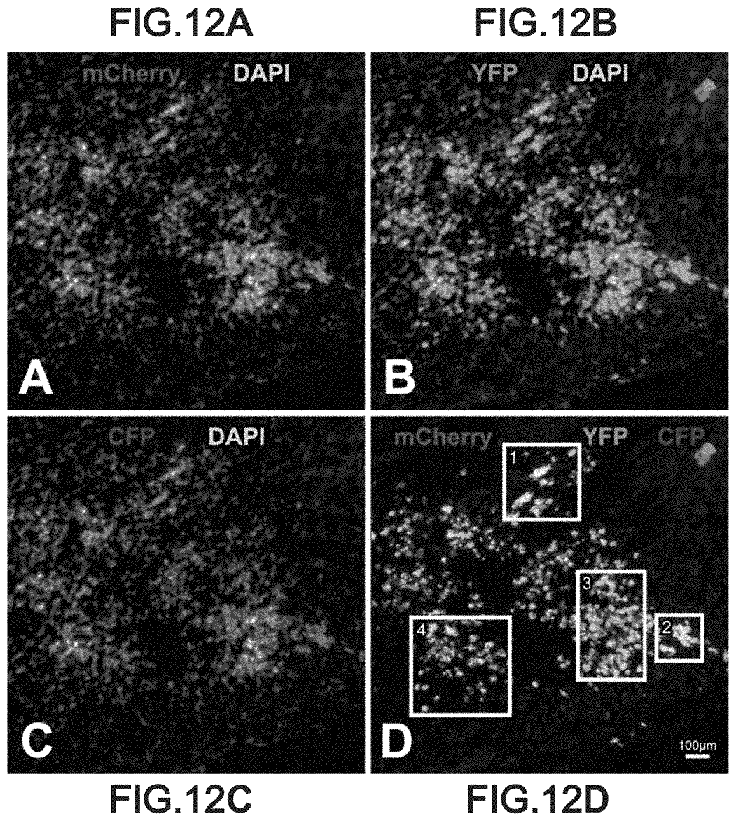

Following acute myocardial infarction, c-kit-BMCs infected with the 3 lentiviruses were delivered to the border zone, and the animals were sacrificed 4-7 (n=12) and 14-21 (n=13) days later. At 4-7 days, areas of myocardial regeneration, varying in size, were identified within the infarcted region of the left ventricular (LV) wall. The foci of tissue repair were characterized by distinct colors, suggesting that clonal expansion of c-kit-BMCs was involved in the process. Individually labeled cells, i.e., red, green or blue, are shown by epifluorescence microscopy in FIGS. 12A (red), 12B (green) and 12C (blue). In the merge panel (FIG. 12D), cell clusters with different colors are found: areas 1 and 2 show white cells, which derived from c-kit-BMCs transduced with the 3 viruses (red, green and blue together=white). Area 3 illustrates predominantly yellow cells, which derived from c-kit-BMCs transduced with 2 viruses (red and green together=yellow). And area 4 illustrates predominantly turquoise cells, which derived from c-kit-BMCs transduced with 2 viruses (blue and green together-turquoise).

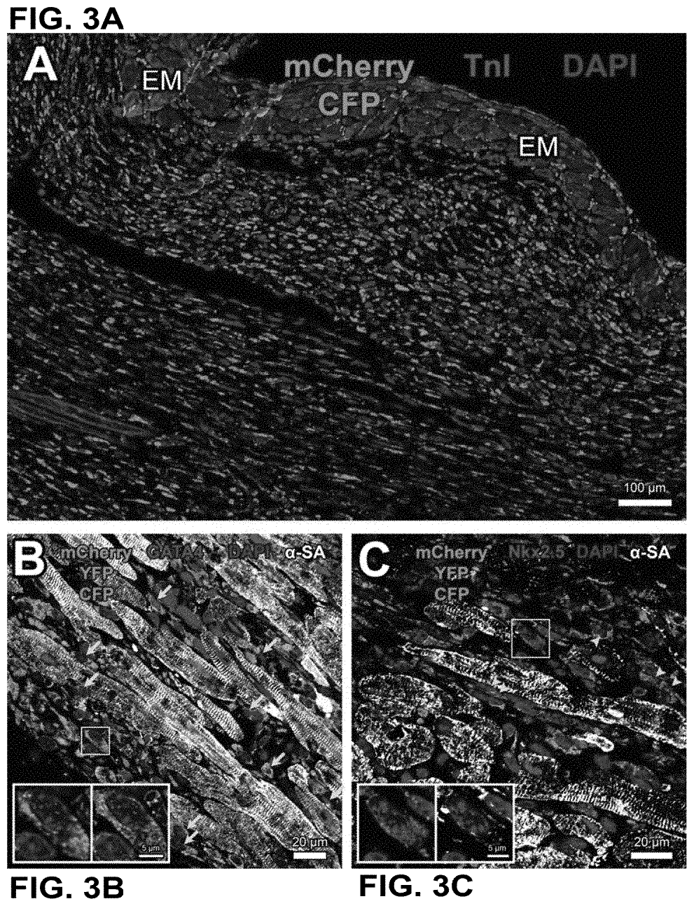

To determine whether the formed cells corresponded to new cardiomyocytes, specific transcription factors and sarcomeric proteins were identified. At 4-7 days after coronary occlusion and cell delivery, the infarcted region was largely replaced by numerous cells positive for mCherry and CFP (green) (FIG. 3A). These cells, mostly elongated in shape, were small in size and resembled forming myocytes. However, cardiac troponin I (cTnI), a marker of mature cardiomyocytes,7 was not detected at this early time point. Occasionally, these developing cardiomyocytes showed some positivity for α-sarcomeric actin (α-SA) and nuclei expressing GATA4 or Nkx2.5 (FIGS. 3B and 3C).

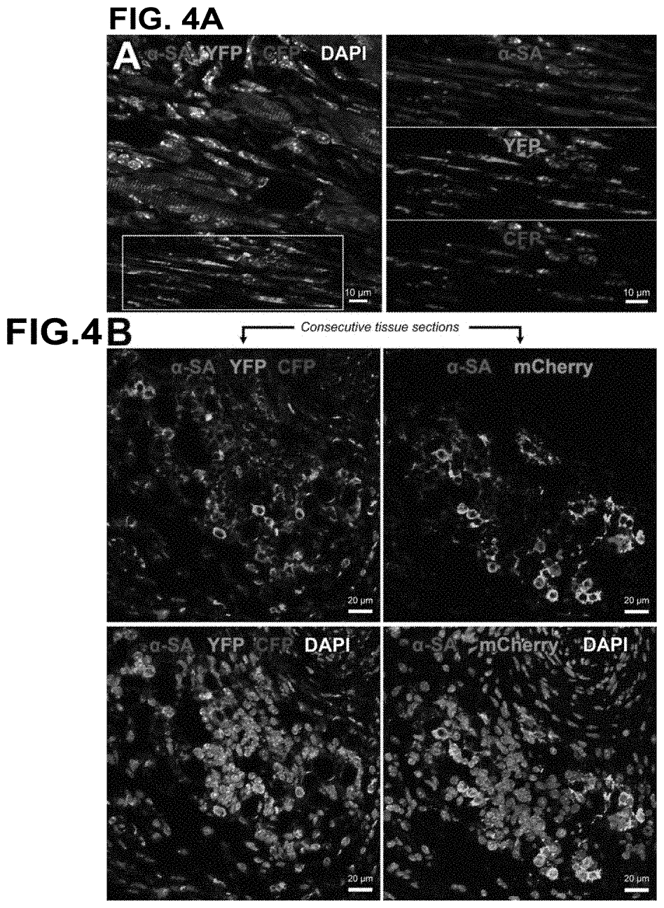

The progeny of c-kit-BMCs carrying YFP (green) and CFP (blue) only, or their combination (turquoise), was apparent in areas of regenerated LV myocardium where α-SA was expressed in some of the cells. Similarly, c-kit-BMCs labeled by YFP (green) and CFP (blue) formed cell clusters positive predominantly for the green tag or both, and α-SA (FIG. 4A). The newly-formed myocardium was evaluated in consecutive sections to identify groups of cells carrying a single vector, i.e., only one color, or two vectors, i.e., two colors combined (FIG. 4B). By this approach, clonal expansion of individual c-kit-BMCs which acquired the cardiomyocyte fate was documented (FIG. 4B). Thus, multicolor clonal marking labels in a distinct manner several categories of c-kit-BMCs and their progeny in vivo.

2.4 Cardiomyocytes and Coronary Vessels are Generated by c-Kit-BMCs

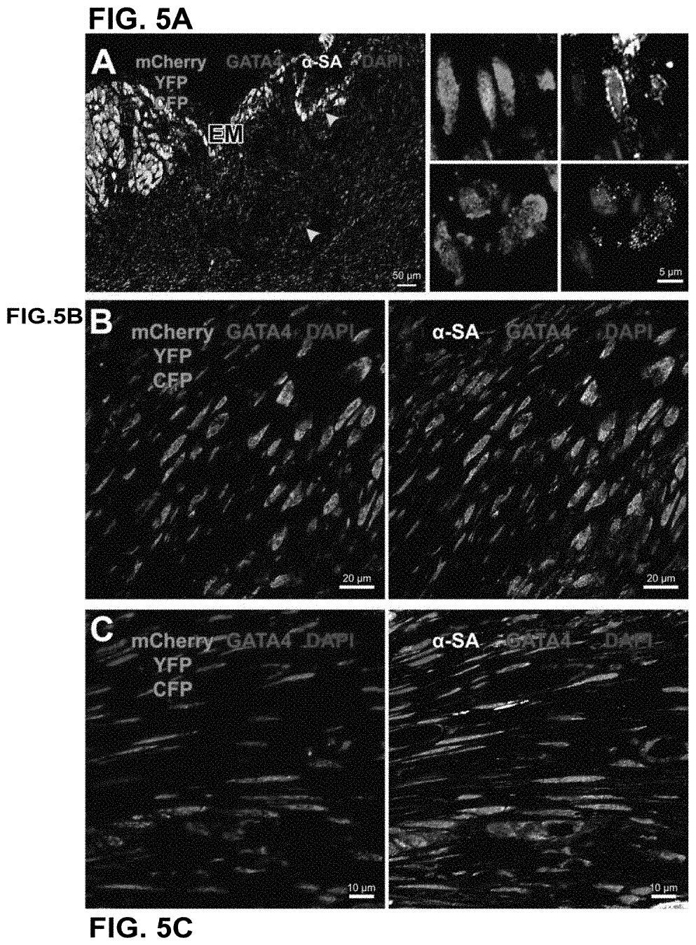

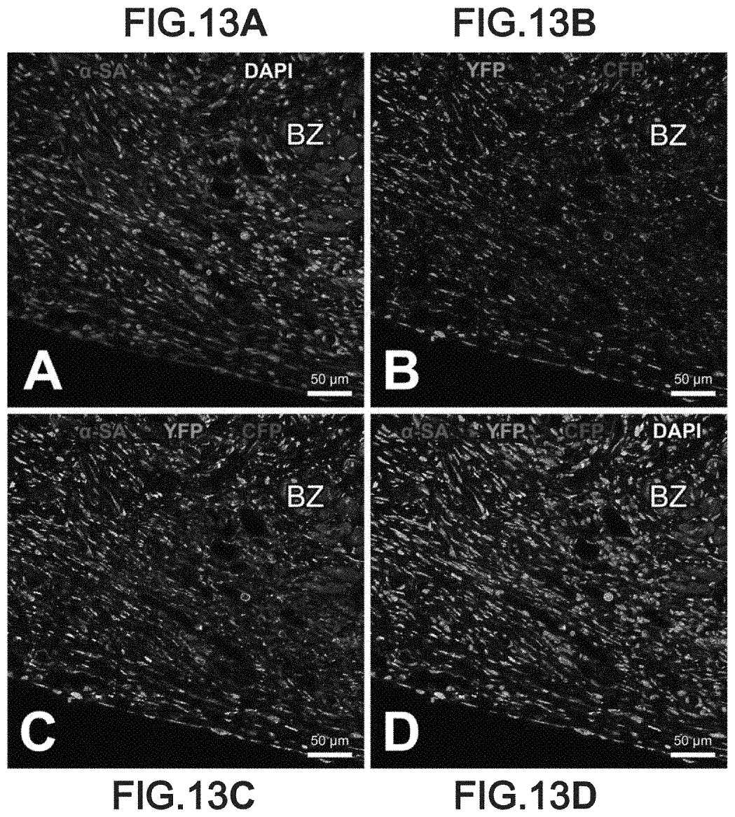

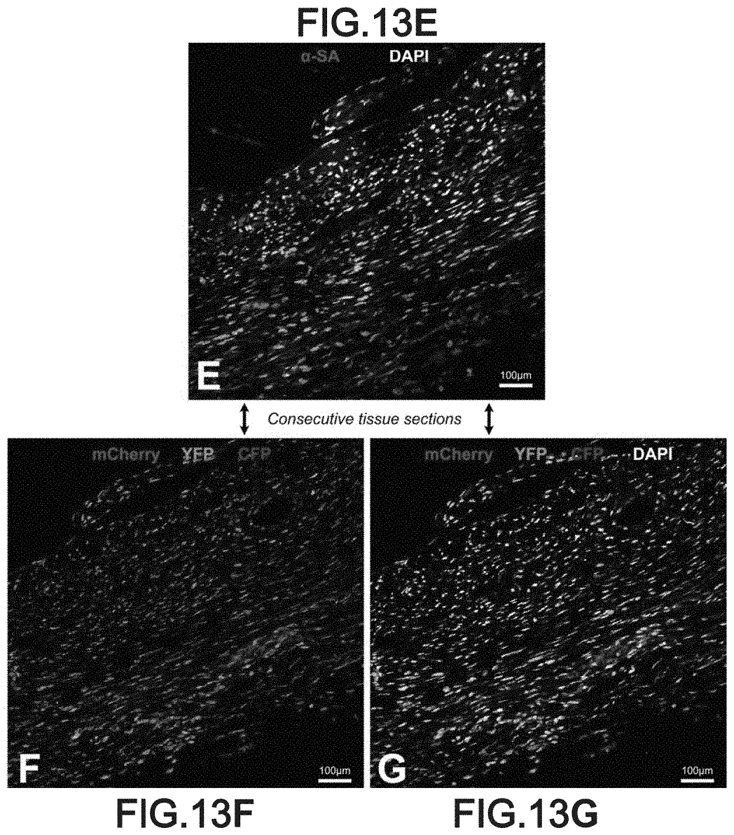

At 14-21 days after infarction and cell delivery, considerable areas of the infarcted LV were replaced by small fluorescently labeled cells, expressing GATA4 (FIG. 5A through 5C). Additionally, cardiomyocytes positive for α-SA were found (FIG. 13A through 13G); YFP (green: panels E-G), CFP (blue: panels E-G) and mCherry (red: panel G) were detected in large clusters of cells. The rather homogeneous distribution of each type of labeling in groups of newly-formed cardiomyocytes suggested that specific c-kit-BMCs were involved in the restoration of the muscle compartment of the LV wall.

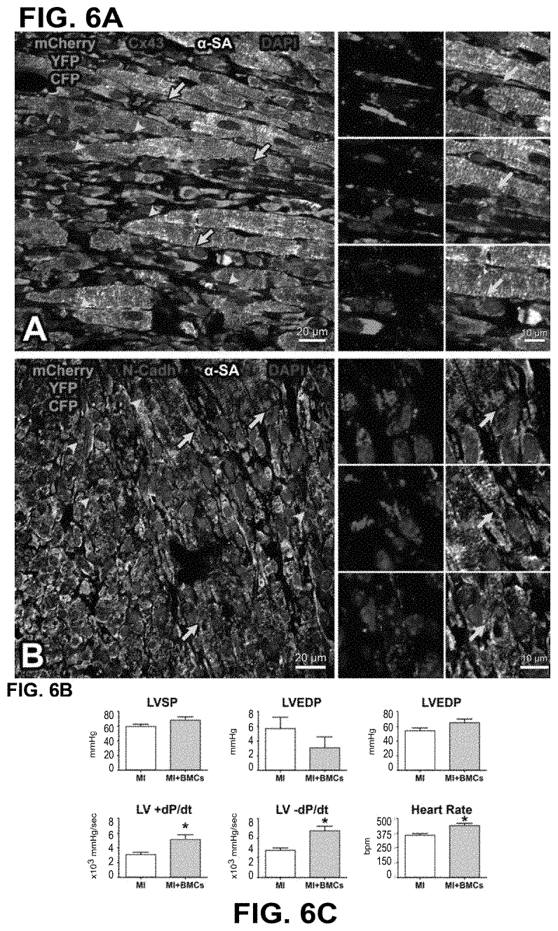

In the 13 cell-treated infarcted hearts, limited regions of new myocardium (not shown) were found together with examples in which, as illustrated in FIGS. 13E, 13F, and 13G, almost the entire necrotic portion of the LV was reconstituted. In all cases, the cardiomyocytes derived from transdifferentiation of c-kit-BMCs showed a rather immature cell phenotype. Whether these small developing cardiomyocytes can acquire adult characteristics chronically remains to be shown. However, at the early and later time points, gap and adherens junctions made by connexin 43 and N-cadherin were present between newly-formed myocytes, and between newly-formed myocytes and spared, recipient myocytes (FIGS. 6A and 6B). The structural integration of pre-existing cardiomyocytes with c-kit-BMCs-derived cardiomyocytes supports the notion that the regenerated cells were coupled with the intact myocardium and contributed to the recovery of the damaged heart.

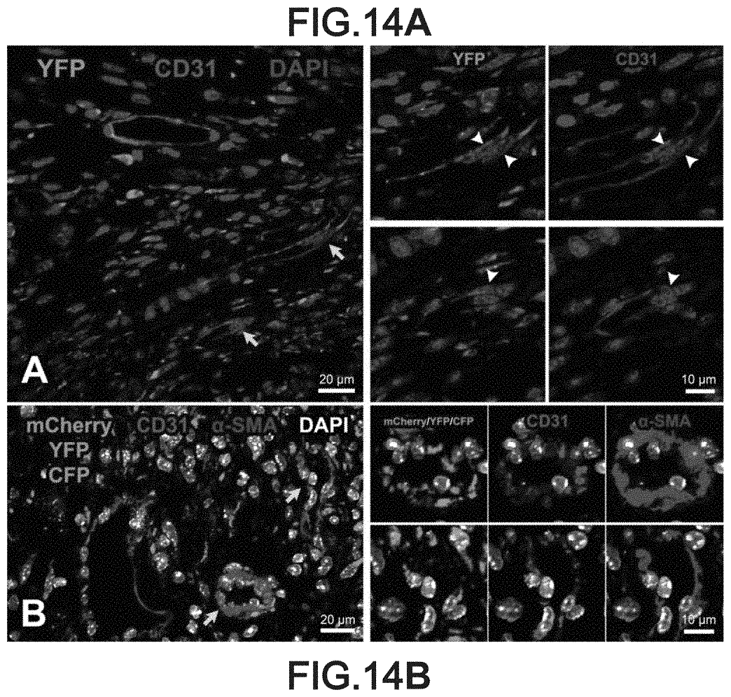

Consistent with the findings obtained by viral gene tagging, c-kit-BMCs acquired in vivo the vascular endothelial and smooth muscle cell phenotypes. Regenerated coronary vessels of different size were identified throughout the reconstituted myocardium (FIGS. 14A and 114B), a fundamental characteristic of effective cardiac repair. Importantly, the myogenic and vasculogenic properties of individual c-kit-BMCs were indicative of their multipotentiality in vivo. Thus, differently tagged c-kit-BMCs and their progeny contribute, in a cooperative manner, to repair the infarcted heart by forming cardiomyocytes and coronary vessels within the recipient myocardium.

2.5 Differentiation of Clonal c-Kit-BMCs In Vivo

Two important observations were made: 1) individual c-kit-BMCs transdifferentiate into cardiac lineages; and 2) the extent of tissue repair varies among animals. Both findings are consistent with previous results in which 40% of infarcted treated mice showed de novo formation of cardiomyocytes and coronary vessels, and c-kit-negative bone marrow cells failed to restore the necrotic myocardium.² These observations raised the possibility that phenotypically distinct populations of c-kit-BMCs have a different capacity to form cardiomyocytes and regenerate the infarcted myocardium.

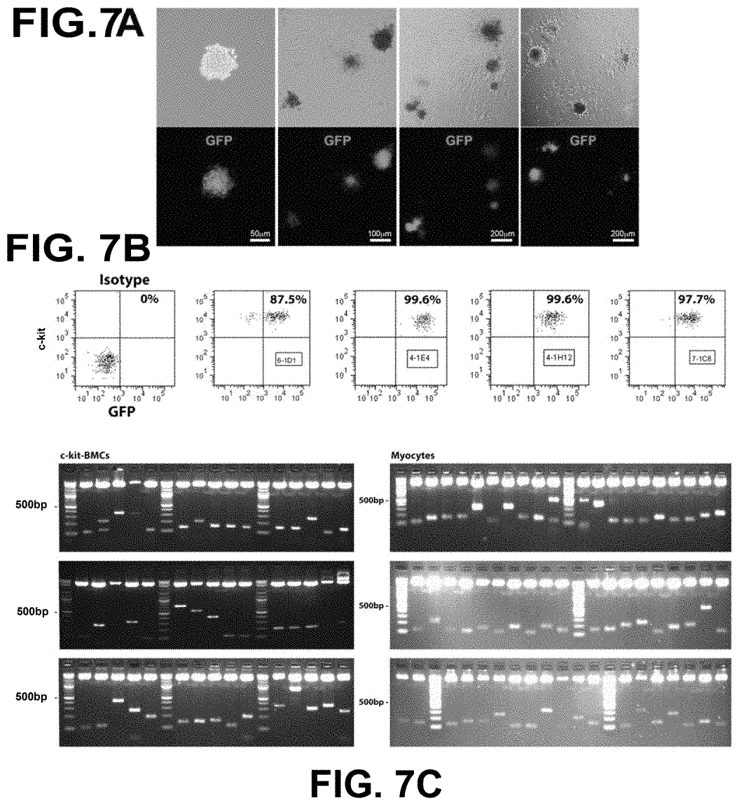

To test this hypothesis with a molecular strategy which is independent from immunolabeling and confocal microscopy and does not allow quantitative assays of tissue formation, freshly isolated c-kit-BMCs were infected with a lentiviral vector carrying GFP. Subsequently, cells were FACS-sorted for c-kit and GFP, and single cells were deposited at limiting dilution in semi-solid medium for clonal growth.⁸ The percentage of c-kit-positive cells in the clones examined by FACS varied from 87.5% to nearly 100% (FIGS. 7A and 7B). Fifteen clones were considered. A total of 1×10⁵ cells, i.e., 2×10⁴ from each of 5 clones, were injected in the border zone of acutely infarcted mice and the animals were sacrificed 21 days later. Three groups of infarcted mice (n=6-8 in each group) were included in this analysis.

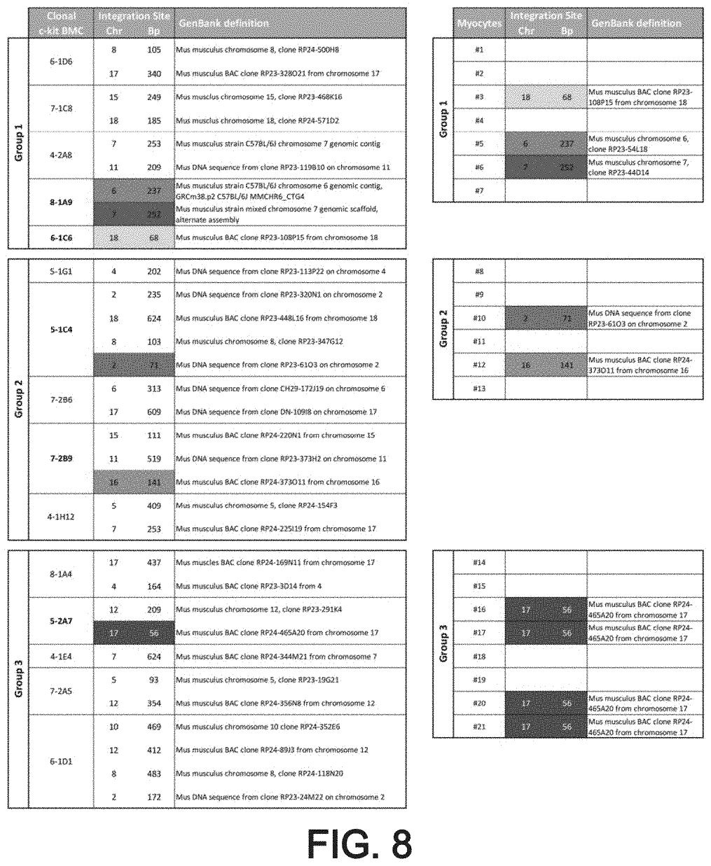

Following enzymatic digestion and cardiomyocyte isolation from 22 hearts, the site of integration of the viral genome in the cardiomyocyte DNA was determined and compared with that present in an aliquot of clonal cells, sampled prior to transplantation from each of the 15 clones utilized for the in vivo studies (FIGS. 7C and 8). When the same site of integration was found in c-kit-BMCs and dissociated cardiomyocytes, clones were defined as myogenic, while clones lacking this association were defined as non-myogenic: of the 15 clones, 5 were myogenic and 10 were non-myogenic (FIG. 8). Despite the fact that only 2×10⁴ cells from each of 5 clones were delivered to the infarcted myocardium of each animal, a common site of integration was found between cardiomyocytes and two of the clones in the first group, two of the clones in the second group and one of the clones in the third group.

Clones from myogenic (n=4) and non-myogenic (n=5) c-kit-BMCs were analyzed by RNA sequencing to define their distinct molecular signature. Freshly isolated c-kit-BMCs (n=5) were also included in this assay. First, the gene expression profile of clonal myogenic and non-myogenic c-kit-BMCs was compared. Only genes showing an expression difference that was statistically significant (P<0.05) were included in the analysis: 1,353 genes were upregulated and 639 were downregulated in myogenic clonal c-kit-BMCs. The differentially expressed genes (DEGs) were then subjected to gene ontology for their functional classification⁹ (Table 1). We found that transcripts of genes involved in cardiac development (Speg, Jag1, Cxadr, Hey2) and muscle cell formation (Speg, Jag1, Cxadr, Hey2, Smyd3, Chrnb1, A1464131) were upregulated in clonal myogenic c-kit-BMCs.

When an expression 2-fold (P<0.05) was considered, five highly scored genes were identified in myogenic c-kit-BMCs: ryanodine receptor 3 (RYR3), Oncostatin M (OSM), Jagged1 (Jag1), Hey2, and SET-dependent methyltransferase 3 (Smyd3). The RYR3 is an intracellular calcium channel implicated in the release of Ca²⁺ from internal stores of muscle cells.¹⁰ OSM is a secreted cytokine involved in the regulation of tissue homeostasis and chronic inflammatory diseases.¹¹ It has been suggested that OSM mediates cardiomyocyte dedifferentiation in vitro and in vivo, upregulates stem cell markers, and improves cardiac function after infarction.¹² Jag1 is the ligand of the Notch receptor, which, upon translocation to the nucleus, upregulates the Hey and Hes family of proteins that act as transcriptional repressors of Notch-dependent genes.¹³ Activation of the Notch1 pathway by Jag1 favors the commitment of cardiac progenitor cells to the myocyte lineage and controls the size of the compartment of transit amplifying myocytes in vitro and in vivo.¹⁴ This function of Notch1 involves the expression of the transcription factor Nkx2.5, which represents a target gene of Notch1 and drives the acquisition of the myocyte lineage of resident cardiac progenitor cells.¹⁴ The function of the Smyd family of proteins in the homeostasis of the adult heart remains to be defined. However, data in the embryonic heart suggest that these methyltransferases are involved in the formation of the myocardium.¹⁵

The contribution of secreted proteins to cardiac repair mediated by bone marrow-derived cells has been emphasized repeatedly. Myogenic clones express increased levels of OSM, which favors cytokine production,¹¹ although DAVID-based gene ontology analysis^{16,17} showed no significant enrichment for cytokine binding, cytokine receptor interaction, cytokine receptor activity and growth factor synthesis in myogenic versus non-myogenic clones. A similar profile was observed in non-myogenic versus myogenic clones. However, the expression of HGF and LIF was upregulated in myogenic clones (Table 2), suggesting that these growth factors may attenuate cardiomyocyte death and promote the migration, division and differentiation of endogenous cardiac progenitor cells.^{18,19} Moreover, VEGF-C, which modulates vascular growth,²⁰ and GDF-6, which is a member of the BMP family of proteins,²¹ were more apparent in non-myogenic clones (Table 3).

When myogenic clonal c-kit-BMCs were compared with freshly isolated c-kit-BMCs, no relevant gene ontology similarities were found. Conversely, significant differences were detected in several classes of genes modulating a variety of physiological processes, including cellular calcium ion homeostasis and transport, regulation of cell migration, proliferation and differentiation, and immune system processes. Thus, myogenic clonal c-kit-BMCs are characterized by a network of developmentally regulated genes reflecting their proficiency to engraft within the environment of the infarcted myocardium,²² transdifferentiate and form cardiomyocytes.¹ Paracrine signals may also be released participating in the regenerative activity of c-kit-BMCs.

TABLE 1
Functional classification of differentially expressed
genes in myogenic and non- myogenic clonal c-kit BMCs.

| | | | FDR | En- | |
|---|---|---|---|---|---|
| GO term | Description | P-value | q-value | richment | Genes |
| GO: | striated | 1.25E−4 | 7.57E−1 | 6.69 | Chrnb1 |
| 0055002 | muscle cell | | | | Speg |
| | development | | | | AI464131 |
| | | | | | Cxadr |
| | | | | | Hey2 |
| | | | | | Smyd3 |
| GO: | muscle cell | 1.25E−4 | 3.78E−1 | 6.69 | Speg |
| 0055001 | development | | | | Chrnb1 |
| | | | | | AI464131 |
| | | | | | Cxadr |
| | | | | | Hey2 |
| | | | | | Smyd3 |
| GO: | regulation of | 3.01E−4 | 6.09E−1 | 2.71 | Srf |
| 0030516 | axon | | | | Ccr5 |
| | extension | | | | Cdkl3 |
| | | | | | Sema5a |
| | | | | | Ntn1 |
| | | | | | Megf8 |
| | | | | | Draxin |
| | | | | | Cdh4 |
| | | | | | Limk1 |
| | | | | | Trpv2 |
| GO: | regulation of | 4.49E−4 | 6.82E−1 | 2.51 | Srf |
| 0061387 | extent of cell | | | | Ccr5 |
| | growth | | | | Cdkl3 |
| | | | | | Sema5a |
| | | | | | Megf8 |
| | | | | | Ntn1 |
| | | | | | Omg |
| | | | | | Draxin |
| | | | | | Cdh4 |
| | | | | | Spg20 |
| | | | | | Limk1 |
| | | | | | Trpv2 |
| GO: | cardiac cell | 5.8E−4 | 7.04E−1 | 8.18 | Speg |
| 0055006 | development | | | | Jag1 |
| | | | | | Cxadr |
| | | | | | Hey2 |
| GO: | response to | 8.75E−4 | 8.85E−1 | 47.22 | Glp2r |
| 0033762 | glucagon | | | | Cd01 |

Gene Ontology (GO) analysis of differentially expressed genes in clonal myogenic and non-myogenic c-kit-BMCs. P-value is uncorrected for multiple testing and FDR q-value is the corrected value using the Benjamini and Hochberg correction. The listed genes were upregulated in clonal myogenic c-kit-BMCs.

TABLE 2
Upregulated genes in clonal myogenic c-kit-BMCs

| | Gene | |
|---|---|---|
| Signaling | Symbol | Gene Name |
| Cytokines | Nlrc4 | NLR family, CARD domain containing 4 |
| | Irf3 | interferon regulatory factor 3 |
| | Il1rap | interleukin 1 receptor accessory protein |
| | Il12rb2 | interleukin 12 receptor, beta 2 |
| | Lipa | lysosomal acid lipase A |
| | Myd88 | myeloid differentiation primary response |
| | | gene 88 |
| Growth Factors | Cntf | Zfp91-Cntf readthrough transcript; zinc finger |
| | | protein 91; ciliary neurotrophic factor |
| | Fgf2 | fibroblast growth factor 2 |
| | Hspe1 | heat shock protein 1 (chaperonin 10); |
| | | predicted gene, EG628438; heat shock |
| | | protein 1 (chaperonin 10), related |
| | | sequence 1; predicted gene 2903 |
| | Hgf | hepatocyte growth factor |
| | Lif | leukemia inhibitory factor |

TABLE 3
Upregulated genes in clonal non-myogenic c-kit-BMCs

| Signaling | Gene Symbol | Gene Name |
|---|---|---|
| Cytokines | Ebi3 | Epstein-Barr virus induced gene 3 |
| | Ccl1 | chemokine (C-C motif) ligand 1 |
| | Cklf | chemokine-like factor |
| | Fbrs | Fibrosin |
| | Gdf6 | growth differentiation factor 6 |
| | Tnfsf10 | tumor necrosis factor (ligand) superfamily, |
| | | member 10 |
| Growth | Fbrs | Fibrosin |
| Factors | Gdf6 | growth differentiation factor 6 |
| | Mdk | Midkine |
| | Pdafa | platelet derived growth factor, alpha |
| | Vegfc | vascular endothelial growth factor C |

### Example 3: Discussion

The results described above relate to the plasticity of c-kit-BMCs and their ability to acquire the cardiomyogenic fate. The population of c-kit-BMCs is diverse and only a subset possesses a molecular signature that favors transdifferentiation and the generation of structures of mesodermal origin distinct from the hematopoietic system. Additionally, c-kit-BMCs may release several cytokines that may have a powerful effect on myocyte survival and the activation of resident progenitor cells with the formation of cardiac muscle and vascular structures.

The likelihood that distinct classes of c-kit-BMCs were employed in various laboratories leading to a variety of divergent results has to be considered. The heterogeneity of stem cells can only be resolved by introducing single-cell-based approaches. In the current study, viral gene tagging and clonal marking were implemented to obtain a molecular confirmation that individual c-kit-BMCs can survive within the infarct and become a relevant component of the cardiac repair process. The recognition that cardiomyocytes, vascular ECs, fibroblasts and c-kit-BMCs isolated from infarcted treated hearts have common sites of viral integration in their genome gives strong evidence in support of bone marrow cell transdifferentiation. c-kit-BMCs commit to the myocyte and vascular lineages, form cardiomyocytes and coronary vessels and self-renew within the tissue possibly having a long-term effect on the recovery of the damaged myocardium.

Understanding the fate specification of stem cells poses serious challenges in view of the high degree of phenotypic and functional heterogeneity encountered in tissue-specific adult stem cells. Despite the shared expression of the c-kit receptor tyrosine kinase, apparently similar c-kit-BMCs behave differently following transplantation in vivo; they can generate myocardial structures or maintain their hematopoietic identity. The variety of hematopoietic stem cells has been documented repeatedly by analyzing surface markers, the molecular profile and the clonal destiny of blood forming cells.²³ The process of cardiomyogenesis was utilized here as readout for the retrospective documentation of the ability of individual c-kit-BMCs to undergo lineage transdifferentiation. The evaluation of clones derived from single c-kit-BMCs was required to define the genes and signaling pathways regulating the properties of these cells in vivo. This methodology allows the identification of rare stem cell subsets, which are lost in population-based studies where they may be viewed as outliers or may be absorbed by larger clusters of cells.

Surface markers that permit the prospective isolation of homogenous stem cell classes with high level of purity have not been discovered yet. The reconstruction of the genealogy of stem cell lineages requires the tracking of single stem cells and their progeny over time.²² In an attempt to characterize the cellular mechanisms involved in the myocardial reconstitution induced by c-kit-BMCs, multicolor clonal marking was employed.⁶ This strategy adheres to the principle that any spectral color can be generated by mixing three primary colors. Based on the additive color theory, seven distinct colors were produced in c-kit-BMCs after their infection with three lentiviral vectors carrying red, green or blue fluorescent protein. As a result, the myocardium generated by the delivery of color-tagged c-kit-BMCs was composed of cells expressing the seven anticipated color possibilities. More importantly, the recognition of uniformly colored clusters of newly-formed specialized cells documented the clonal expansion and differentiation of individual c-kit-BMCs in vivo. Comparable findings were obtained with viral gene tagging which, together with multicolor clonal marking, demonstrate the polyclonal origin of myocardial repair.

The data described herein strongly suggest that a class of adult c-kit-BMCs implanted in the infarcted heart loses the hematopoietic fate and integrates within the host myocardium, adopting the cardiac destiny. The prevailing belief, however, is that bone marrow progenitor cells lack this fundamental ability, and the recovery of the injured myocardium promoted by the delivered cells occurs exclusively via paracrine signals, which activate resident stem/progenitor cells.²⁵ As documented here, c-kit-BMCs have a dual modality of action since they possess a molecular signature that comprises a network of cardiopoietic genes and transcripts for multiple growth factors, which are differentially expressed in myogenic and non-myogenic clonal cells. Thus far, only BM-MNCs, CD34-positive cells and mesenchymal stromal cells have been employed clinically.³ The findings herein indicate that c-kit-BMCs may be a more successful form of cell therapy for the failing heart, an alternative to be considered in view of the limited beneficial effects observed with BM-MNCs experimentally²⁶ and clinically.³ The possibility that c-kit-BMCs may fuse with recipient cardiomyocytes prior to myocardial regeneration²⁵ cannot be excluded by viral gene tagging. But, the upregulation of developmentally regulated cardiac genes in c-kit-BMCs, the fetal-neonatal characteristics of newly-formed cardiomyocytes, and the previous analysis of this process,²⁸ make this an unlikely event.

Small double-blind multicenter clinical trials in which BM-MNCs have been administered to patients with acute and chronic ischemic heart failure have been completed.²⁹ Despite positive results, albeit modest, the mechanism by which BM-MNCs improve the outcome of acute myocardial infarction and chronic ischemic cardiomyopathy in humans remains unclear. Currently, a large clinical trial is in progress (ClinicalTrials.gov Identifier: NCT01569178), but uncertainties persist about the actual impact of BM-MNCs on the decompensated heart and patient mortality. None of the clinical trials performed thus far has employed c-kit-BMCs, and caution should be exercised in assuming that BM-MNCs have the characteristics of hematopoietic progenitors.

Although several laboratories have tested the potential therapeutic efficacy of c-kit-BMCs and resident c-kit-positive cardiac progenitor cells (c-kit-CPCs),^{18,25,26,30-37} whether c-kit-BMCs are inferior, equal or superior to c-kit-CPCs for myocardial repair has never been tested. Based on a microarray assay, these two classes of c-kit-positive cells have a highly distinct transcriptional profile,³⁸ but when delivered to the same microenvironment appear to acquire similar functional characteristics. The molecular differences may be attenuated within the damaged myocardium and bone marrow-derived and cardiac-derived progenitor cells act similarly in reconstituting partly the integrity of the tissue. In analogy to c-kit-BMCs, c-kit-CPCs have been found recently to operate only via paracrine mechanisms³⁷ or to be able to differentiate into cardiomyocytes and coronary vessels and concurrently exert a paracrine effect on the recipient heart.³⁹ It is not surprising that despite the accuracy and sophisticated methodologies employed by different research groups diverse results are obtained. The approach implemented in the current study may provide a strategy that may help clarifying these apparent discordant observations. However, what is consistent is the beneficial impact of c-kit-positive cells on the myocardial structure and function of the injured heart.

Collectively, c-kit-BMCs constitute a critically important hematopoietic stem cell class; a subpopulation of these cells has the intrinsic ability to cross lineage boundaries and commit to the cardiac fate. Whether the same or other c-kit-BMC categories can differentiate into lung epithelial cells⁴⁰ or neural cells has been proposed in the past, but the potential clinical translation of these interesting observations has not occurred. However, the c-kit-BMCs characterized herein have significant implications for the management of the post-infarcted human heart.

### REFERENCES

1. Orlic D, Kajstura J, Chimenti S, Jakoniuk I, Anderson S M, Li B, Pickel J, McKay R, Nadal-Ginard B, Bodine O M, Leri A. Anversa P. Bone marrow cells regenerate infarcted myocardium. Nature. 2001.410:701-705.
2. Sanada F, Kim J, Czarna A, Chan N Y, Signore S, Ogórek B, Isobe K, Wybieralska E, Borghetti G, Pesapane A, Sorrentino A, Mangano E, Cappetta D, Mangiaracina C, Ricciardi M, Cimini M, Ifedigbo E, Perrella M A, Goichberg P, Choi A M, Kajstura J, Hosoda T, Rota M, Anversa P, Leri A. c-Kit-positive cardiac stem cells nested in hypoxic niches are activated by stem cell factor reversing the aging myopathy. Gire Res. 2014; 114:41-55.
3. Yonemura Y, Ku H, Lyman S D, Ogawa M. In vitro expansion of hematopoietic progenitors and maintenance of stem cells: comparison between FLT3/FLK-2 ligand and KIT ligand. Blood. 1997; 89:1915-1921. 150] 4. Gude N, Joyo E, Toko H, Quijada P, Villanueva M, Hariharan N, Sacchi V, Truffa S, Joyo A, Voelkers M, Alvarez R, Sussman M A. Notch activation enhances lineage commitment and protective signaling in cardiac progenitor cells. Basic Res Cardiol. 2015; 110:29.
5. Hosoda T, D'Amario D, Cabral-Da-Silva M C, Zheng H, Padin-Iruegas M E, Ogorek B, Ferreira-Martins J, Yasuzawa-Amano S, Amano K, Ide-Iwata N, Cheng W, Rota M, Urbanek K, Kajstura J, Anversa P, Leri A. Clonality of mouse and human cardiomyogenesis in vivo. Proc Natl Acad Sci USA. 2009; 106:17169-17174.
6. Ellison G M, Vicinanza C, Smith A J, Aquila I, Leone A, Waring C D, Henning B J, Stirparo G G, Papait R, Scarfo M, Agosti V, Viglietto G, Condorelli G, Indolfi C, Ottolenghi S, Tarella D, Nadal-Ginard B. Endogenous cardiac stem cell activation by insulin-like growth factor-1/hepatocyte growth factor intracoronary injection fosters survival and regeneration of the infarcted pig heart. J Am Coll Cardiol. 2011; 58:977-986.
7. Hui E K-W, Wang P-C, Lo S. J. PCR-Based strategies to clone unknown DNA regions from known foreign integrants: an overview. In: Chen B-Y, Janes H W, eds. PCR Cloning Protocols, Totowa, N.J.: Humana Press; 2000. p 249-274.
8. Schmidt M, Glimm H, Lemke N, Muessig A, Speckmann C, Haas S, Zickler P, Hoffmann G, Von Kalle C. A model for the detection of clonality in marked hematopoietic stem cells. Ann NY Acad Sci. 2001; 938:146-155.
9. Dunbar C E. Stem cell gene transfer: insights into integration and hematopoiesis from primate genetic marking studies. Ann NY Acad Sci. 2005; 1044: 178-182.
10. Weber K, Thomaschewski M, Benten D, Fehse B. RGB marking facilitates multicolor clonal cell tracking. Nat Med. 2011; 17:504-509.
11. Weber K, Thomaschewski M, Benten D, Fehse B. RGB marking with lentiviral vectors for multicolor clonal cell tracking. Nat Protoc. 2012:7:839-849.
12. Cornils K, Thielecke L, Huser S, Forgber M, Thomaschewski M, Kleist N, Hussein K, Riecken K, Volz T, Gerdes S, Glauche I, Dahl A, Dandri M, Roeder I, Fehse B. Multiplexing clonality: combining RGB marking and genetic barcoding. Nucleic Acids Res. 2014; 42:e56.
13. Gude N A, Emmanuel G, Wu W, Cottage C T, Fischer K, Quijada P, Muraski J A, Alvarez R, Rubio M, Schaefer E, Sussman M A. Activation of Notch-mediated protective signaling in the myocardium. Gire Res. 2008, 102:1025-1035. 160] 14. Signore S, Sorrentino A, Borghetti G, Cannata A, Meo M, Zhou Y, Kannappan R, Pasqualini F, O'Malley H, Sundman M, Tsigkas N, Zhang E, Arranto C, Mangiaracina C, Isobe K, Sena B F, Kim J, Goichberg P, Nahrendorf M, Isom L L, Leri A, Anversa P, Rota M. Late Na(+) current and protracted electrical recovery are critical determinants of the aging myopathy. Nat Commun. 2015; 6:8803.
15. Meo M, Meste O, Signore S, Sorrentino A, Cannata A, Zhou Y, Matsuda A, Luciani M, Kannappan R, Goichberg P, Leri A, Anversa P, Rota M. Reduction in Kv current enhances the temporal dispersion of the action potential in diabetic myocytes: insights from a novel repolarization algorithm. J Am Heart Assoc. 2016; 5:e003078.
16. McGettigan P A. Transcriptomics in the RNA-seq era. Curr Opin Chem Biol. 2013, 17:4-11.
17. Trapnell C, Williams B A, Pertea G, Mortazavi A, Kwan G, van Baren M J, Salzberg S L, Wold B J, Pachter L. Transcript assembly and quantification by RNA-Seq reveals unannotated transcripts and isoform switching during cell differentiation. Nat Biotech 2010; 28:511-515.

What is claimed is:

1. A method of treating or preventing a heart disease or disorder in a subject in need thereof comprising administering isolated myogenic bone marrow cells to the subject, wherein the myogenic bone marrow cells are c-kit positive (c-kit-BMCs).

2. The method of claim 1, wherein the heart disease or disorder is heart failure, diabetic heart disease, rheumatic heart disease, hypertensive heart disease, ischemic heart disease, cerebrovascular heart disease, inflammatory heart disease and/or congenital heart disease.

3. The method of claim 1, wherein the c-kit-BMCs are a subpopulation of c-kit positive bone marrow cells isolated from bone marrow.

4. The method of claim 1, wherein the c-kit-BMCs are able to transdifferentiate into cardiomyocytes, endothelial cells, fibroblasts, coronary vessels and/or cells of mesodermal origin.

5. The method of claim 1, wherein the c-kit-BMCs have enhanced expression of cardiopoietic genes compared to non-myogenic c-kit positive bone marrow cells.

6. The method of claim 1, wherein the c-kit-BMCs have enhanced expression of RYR3, OSM, Jag1, Hey2 and Smyd3 compared to non-myogenic c-kit positive bone marrow cells.

7. A method of repairing and/or regenerating damaged tissue of a heart in a subject in need thereof comprising:
(a) extracting c-kit positive bone marrow cells from bone marrow;
(b) selecting myogenic c-kit positive bone marrow cells (c-kit-BMCs) from step (a);
(c) culturing and expanding said c-kit-BMCs from step (b); and
(d) administering a dose of said c-kit-BMCs from step (c) to an area of damaged tissue in the subject effective to repair and/or regenerate the damaged tissue of the heart.

8. A method of producing myogenic c-kit positive bone marrow cells (c-kit-BMCs), comprising:
(a) isolating c-kit positive bone marrow cells from bone marrow;
(b) selecting myogenic c-kit positive bone marrow cells (c-kit-BMCs) from step (a); and
(c) culturing and expanding the c-kit-BMCs of step (b), thereby producing c-kit-BMCs.

9. The method of claim 7, wherein the selecting step comprises selecting c-kit-BMCs having enhanced expression of RYR3, OSM, Jag1, Hey2 and Smyd3.

10. The method of claim 9, wherein the selecting step comprises selecting c-kit-BMCs having enhanced expression of RYR3, OSM, Jag1, Hey2 and Smyd3.
